SEQUENCE LISTING (2 sequences)

SEQ ID NO: 1 - DNA, 21 bp, Oryza sativa
ggttgggcaggccagcgtatc

SEQ ID NO: 2 - DNA, 21 bp, oryza sativa
ccaatgcctaaagagaggtta

(19) United States
(12) Patent Application Publication
(10) Pub. No.: US 2010/0186107 A1
(43) Pub. Date: Jul. 22, 2010

(54) METHODS OF PRODUCING IMPROVED FRAGRANT PLANTS

(75) Inventors: Timothy Liam FITZGERALD, Goonellabah (AU); Daniel Lex Ean WATERS, Goonellabah (AU); Robert James HENRY, Tuckombil (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: GRAIN FOODS CRC LTD, North Ryde (AU)

(21) Appl. No.: 12/686,917

(22) Filed: Jan. 13, 2010

Related U.S. Application Data
(60) Provisional application No. 61/144,292, filed on Jan. 13, 2009.

(51) Int. Cl. A01H 5/00; A01H 1/02; C12N 15/82

(52) U.S. Cl. 800/275; 800/260; 800/298; 800/278

(57) ABSTRACT

Methods of producing fragrant plants with improved crop performance wherein the fragrant plant has or retains at least some fragrance are provided. Typically, the methods include selecting for or conferring at least partial tolerance to one or more abiotic stress factors. The methods of the invention may be useful to confer at least partial tolerance to salt. The methods of the invention produce fragrant plants that exhibit an improvement in the ability to perform as a crop.

### CROSS-REFERENCE TO RELATED APPLICATIONS

This patent application claims the benefit of U.S. Provisional Patent Application No. 61/144,292, filed Jan. 13, 2009.

### INCORPORATION-BY-REFERENCE OF MATERIAL SUBMITTED ELECTRONICALLY

Incorporated by reference in its entirety herein is a computer-readable nucleotide/amino acid sequence listing identified as follows: One 575 Byte ASCII (Text) file named “SequenceListing-706003.TXT,” created on Jan. 13, 2010.

### FIELD OF THE INVENTION

This invention relates to methods for producing fragrant plants. More particularly, this invention relates to method for producing fragrant plants with improved crop performance.

### BACKGROUND TO THE INVENTION

Salt stress has a significant impact on the yield of all major cereal crops. Rice (Oryza sativa) is a salt sensitive species (Koyam et al., 2001) with most varieties suffering significant reduction in growth when the concentration of salt is greater then 10 mM. Proline accumulation is associated with salt tolerance in many plant species, including rice (Garcia et al., 1997). Glycinebetaine is a secondary metabolite derived from proline which is a highly effective protectant against osmotic stress (Le Rudelier et al, 1984), salinity stress (Harinasut et al., 1996) and temperature stress (Eung-Jun et al., 2004; Yang et al., 2005) (for review see Gorham, 1995). In a number of species, betaine aldehyde dehydrogenase (BADH) works in conjunction with choline monooxygenase (CMO) to produce glycinebetaine (GB). Most plants appear to possess two BADH homologs each of which have been shown to play a role in drought and salinity stress responses in cereals (Ishitani et al., 1995; Wood et al., 1996).

Rice possesses two BADH homologs, BADH1 and BADH2 (Bradbury et al., 2005). The gene for BADH1 is located on chromosome four whilst the gene for BADH2 is located on chromosome eight (Cordeiro et al., 2002; Jin et al., 2003; Bradbury et al., 2005). In rice BADH1 and BADH2 share 75% identity at the amino acid level with the catalytic domains sharing 92% similarity. Both appear to be targeted to the peroxisome, since each possesses an SKL signal at the C-terminus (Nakamura et al., 2001). Despite possessing BADH1 and BADH2, rice does not accumulate GB (Rathinasabapathi et al., 1993), probably because CMO is non-functional in rice (Shirasawa et al., 2006). Although rice does not accumulate GB, BADH1 transcript levels increase in salt stressed Japonica and Indica non-fragrant rice varieties (Nakamura et al., 1997) indicating that rice BADH1 is likely to be involved in the salt stress response. If this is the case, it must occur by a yet to be determined mechanism which does not involve GB.

The compound 2-acetyl-1-pyrroline (2AP) is associated with the aroma of many foods (Bradbury et al., 2005), however, it is most closely associated with the fragrance of basmati and jasmine style rice varieties. Although non-fragrant rice varieties also accumulate 2AP, it is at concentrations which are much lower than those found in fragrant rice (Widjaja et al., 1996). In 2005 Bradbury et al. discovered the gene which is primarily responsible for rice fragrance. Interestingly, this gene was the BADH homolog BADH2. All fragrant and non-fragrant rice varieties studied to date possess a BADH1 gene which produces a functional enzyme when expressed in Escherichia coli (E. coli). In contrast, non-fragrant rice varieties possess a functional BADH2 allele, whilst fragrant rice varieties possess a BADH2 allele which contains a premature termination codon (PTC) and produces an enzyme that is non-functional when expressed in E. coli. It is proposed that the truncated BADH2 enzyme leads to the accumulation of 2AP by a yet to be confirmed biochemical mechanism (Bradbury et al., 2005).

BADH2 transcript levels are up to five times greater than BADH1 transcript levels in leaf, developing seed and mature seed from non-fragrant rice varieties. This indicates that fragrant rice varieties possess significantly lower amounts of functional BADH gene transcript than fragrant varieties. Despite this, no loss of abiotic stress tolerance in fragrant rice varieties has been reported, although it is known that current fragrant rice varieties produce approximately 40% less yield than non-fragrant varieties. Whilst BADH1 transcript levels exhibit a consistent increase in response to salt treatment in leaf tissue from fragrant and non-fragrant rice varieties, BADH2 transcript levels do not appear to respond. Based on these results it was suggested that BADH2 does not play a role in abiotic stress tolerance in rice, providing an explanation for why no less in tolerance to abiotic stress has been reported in fragrant rice varieties possessing non-functional BADH2.

### SUMMARY OF THE INVENTION

Fragrant plant varieties, and in particular fragrant grain varieties, represent significant commercial value. However these fragrant plant varieties can display decreased yield and performance compared to non-fragrant varieties, probably due in part, to a lack of abiotic stress tolerance. Thus, there exists a need for fragrant plant varieties with increased stress tolerance which, in turn, should result in positive outcomes on fragrant plant crop performance.

Therefore the invention is broadly directed to utilising a genetic association between the fragrance phenotype in fragrant grain varieties and decreased tolerance to abiotic stresses in order to improve the crop yield and/or performance of fragrant plant varieties.

In one broad form, the invention is directed to a method of producing a fragrant plant with improved crop performance which has or retains at least some fragrance. In one embodiment, the fragrant plant with improved crop performance is produced using conventional plant breeding. In another embodiment, the fragrant plant with improved crop performance is produced using recombinant DNA technology. In yet other embodiments, the fragrant plant with improved crop performance is produced by mutagenising a fragrant plant to generate genetic diversity to thereby produce a fragrant plant with improved crop performance.

In a first aspect, the invention provides a method of producing a fragrant plant with improved crop performance, said method including the step of propagating a fragrant plant with improved crop performance from a fragrant plant and a plant which has, or has been selected for, at least partial tolerance to one or more abiotic stress factors, to thereby produce a fragrant plant with improved crop performance.

In a second aspect, the invention provides a method of producing a genetically-modified fragrant plant with improved crop performance, said method including the step of genetically modifying one or more fragrant plant cells or tissues to thereby produce a genetically-modified fragrant plant with improved crop performance, wherein said genetically-modified fragrant plant with improved crop performance retains at least a portion of a fragrance.

It is envisaged that in particular embodiments, the step of genetic modification encompasses introduction of a heterologous nucleic acid into one or more fragrant plant cells or tissues.

Preferably, the heterologous nucleic acid is not codA.

In a third aspect, the invention provides a method of producing a fragrant plant with improved crop performance, said method including the steps of:

(i) introducing one or more mutations into genetic material of a fragrant plant; and

(ii) selecting a fragrant plant having one or more mutations which is at least partially tolerant to one or more abiotic stress factors,
to thereby produce a fragrant plant with improved crop performance.

In a fourth aspect, the invention provides a fragrant plant with improved crop performance produced according to any method of the aforementioned aspects.

In a fifth aspect, the invention provides a genetically-modified fragrant plant with improved crop performance, wherein the genetically-modified fragrant plant is at least partially tolerant to one or more abiotic stress factors.

In a sixth aspect, the invention provides a method of producing a fragrant plant with improved crop performance, said method including the step of propagating a fragrant plant with improved crop performance from at least two fragrant plants with improved crop performance produced according to any one of the methods of the aforementioned aspects.

In preferred embodiments, the one or more abiotic stress factors are selected from the group consisting of salt, drought, cold, freezing, high temperature, anoxia, high light intensity, nutrient imbalance and heavy metal tolerance.

More preferably, the abiotic stress factor is salt.

Preferably, the plant or the fragrant plant is a monocotyledonous plant or a dicotyledonous plant.

More preferably the plant or fragrant plant is selected from the group consisting of asparagus, bamboo, corn, barley, wheat, rice, sorghum, onion, pearl millet, rye, oats, rape, Zea mays, Zoysia tenuifolia, Musa acuminata, Pandan, tomato, beans, soybeans, peppers, lettuce, peas, alfalfa, cabbage, tobacco, broccoli, cauliflower, brussel sprouts, radish, carrot, beets, eggplant, spinach, cucumber, squash, sunflowers and combinations thereof.

Even more preferably, the plant or the fragrant plant is selected from the group consisting of wheat, rice, barley, oats, maize, Zea mays, sorghum, Zoysia tenuifolia and combinations thereof.

In preferred embodiments, the plant or fragrant plant is rice. Preferably, the rice variety is selected from the group consisting ofjasmine, Basmati 370, Kyeema, Dellmont, Dragon's Eyeball 100, Dumsorhk, Khao Dawk Mali 105, Goolarah, Gobindobhog, Pokkali, KDML and combinations thereof.

Also contemplated are cells, tissues, leaves, fruit, flowers, seeds and other reproductive material, material useful for vegetative propagation, progeny plants including F1 hybrids, male-sterile plants and all other plants and plant products derivable from said plants, and preferably said genetically-modified plants.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

The patent or application file contains at least one drawing executed in color. Copies of this patent or patent application publication with color drawing(s) will be provided by the Office upon request and payment of the necessary fee.

In order that the invention may be readily understood and put into practical effect, preferred embodiments will now be described by way of example with reference to the accompanying figures wherein like reference numerals refer to like parts and wherein:

FIG. 1A: Fragrant individuals photographed post-harvest after two days drying. Largest (by mass) of the two individuals grown for each variety at each level of salt treatment shown. A: Dumsorhk, B: Basmati 370, C: Goolarah, D: Dragon' s Eyeball 100, E: Kyeema, F: Dellmont, G: Jasmine. * Indicates that only a single individual grew past the seedling stage. B: Non-fragrant individuals photographed post-harvest after two days drying. Largest (by mass) of the two individuals grown for each variety at each level of salt treatment shown. A: Jarrah, B: Amaroo, C: Millin, D: Doongara, E: Calrose, F: Teqing. * Indicates that only a single individual grew past the seedling stage. Images are to scale, on average the fragrant varieties studied were taller then the non-fragrant varieties.

FIG. 2 Seed produced by fragrant and non-fragrant rice varieties at each level of salt treatment. Seed produced by the highest yielding individual for each variety at each salt level is shown. * Indicates that only a single individual grew past the seedling stage.

FIG. 3 Relative transcript levels of BADH1 and BADH2 in mature seed from the non-fragrant rice varieties Teqing and Millin grown at two levels of salt treatment: 0 mM and 17 mM additional salt.

FIG. 4 Biochemical pathway leading to GABA via functional BADH2 as proposed by Bradbury et al. (2008). It is well known that Proline increases under conditions of abiotic stress. The present inventors have shown that BADH2 gene expression is increased in mature seed from non-fragrant rice grown under salt treatment. It is proposed that BADH2 in non-fragrant rice varieties assists in salt tolerance via the production of GABA and that the loss of salt tolerance observed in fragrant rice varieties occurs due to their inability to accumulate GABA via BADH2.

### DETAILED DESCRIPTION OF THE INVENTION

The present invention is predicated, at least in part, on the finding by the present inventors of a linkage between a gene responsible for fragrance and the susceptibility to salt stress of such fragrant varieties. More particularly, the present inventors have demonstrated that fragrant rice varieties subjected to high salt treatment have a reduced capacity to produce mature seed compared to non-fragrant rice varieties under the same salt conditions. More particularly, the loss of stress tolerance in fragrant genotypes is linked to or associated with the betaine aldehyde dehydrogenase (BADH) gene and more particularly, the BADH2 gene. As shown in the Examples section, BADH2 gene expression is increased in mature seed from non-fragrant rice grown under salt treatment. Without being limited to any particular theory, it is proposed that BADH2 in non-fragrant rice varieties assists in salt tolerance via the production of GABA and that the loss of salt tolerance observed in fragrant rice varieties occurs due to their inability to accumulate GABA via BADH2.

Moreover, the present invention exploits, in part, the salient finding that fragrant plant varieties are more susceptible to salt than non-fragrant varieties which, in turn, may result in deleterious effects on yields, field performance and crop performance of fragrant varieties.

Therefore the present invention broadly provides methods for producing a fragrant plant with improved crop performance wherein the fragrant plant retains or has at least a portion of a fragrance.

As used herein, by “crop performance” is meant the ability of a plant, or part thereof, to produce a crop under normal field conditions. In the context of the present invention, “crop performance” includes one or a combination of parameters, and in particular agronomic and/or agricultural parameters such as plant growth, plant yield, seed or cereal grain yield, fruit yield, milling yield, plant vigour and quality of plants and parts thereof such as fruit, although without limitation thereto. It will be appreciated that what constitutes normal field conditions is dependent on the plant of interest. For example, normal field conditions for rice is a semi-aquatic environment whereas normal field conditions for wheat is a dry and hot terrestrial environment.

By “crop” is meant agricultural produce or cultivated produce, either whilst growing or when gathered.

The term “improved”, “improvement” and “improve” in the context of the present invention relates, in general, to superior or enhanced crop performance of a fragrant plant relative to what is normally expected in the absence methods of selection and breeding, genetic modification and/or introduction of mutations of the present invention as described herein. For example, an improvement in crop performance may manifest as one or a plurality of outcomes such as an increase in seed yield or alternatively, an increase in the ability of a plant to tolerate abiotic stress, but is not limited thereto. Improved crop performance may relate to not only improvements in individual plants but is inclusive of improvements to the crop performance of an overall population of a crop of plants.

By “plant” is meant a fragrant and/or a non-fragrant plant and is inclusive of non-fragrant plants capable of producing a fragrance. Suitable plants include monocotyledonous or dicotyledonous plants, and is inclusive of crop plants. Examples of monocotyledonous plants include asparagus, bamboo, corn, barley, wheat, rice, sorghum, onion, pearl millet, rye, oats, rape, Zoysia tenuifolia, Musa acuminata and Pandan, although without limitation thereto. Non-limiting examples of dicotyledonous plants include tomato, beans, soybeans, peppers, lettuce, peas, alfalfa, cabbage, broccoli, cauliflower, brussel sprouts, raddish, carrot, beets, eggplant, spinach, cucumber, squash and sunflowers. Preferably, the plants are selected from the group consisting of wheat, rice, barley, oats, maize, sorghum and Zoysia tenuifolia. In particularly preferred embodiments, the plant or fragrant plant is rice. In certain embodiments, the rice variety is selected from the group consisting of jasmine, Basmati 370, Kyeema, Dellmont, Dragon's Eyeball 100, Dumsorhk, Khao Dawk Mali 105, Goolarah, Gobindobhog, Pokkali and KDML.

The term “plant part” refers to plant components such as grains, flowers, seeds and other reproductive material such as ova, leaves, skin, pollen, roots, kernel, stem, bracts, branches, fruit, material useful for vegetative propagation, and may also refer to cells and/or tissues (e.g., from a protoplast, callus, or tissue part).

Throughout this specification, by “fragrant” or “fragrance” is meant the aroma, taste or smell resulting from one or more fragrant molecules that are produced by fragrant plant varieties such as Jasmine rice or Khao Dawk Mali (KDML) 105 rice but are not produced or a produced at a level of which is below the threshold level of detection by non-fragrant plant varieties. Non-limiting examples of one or more fragrance molecules which can give rise to fragrance or aroma include 2-acetyl-1-pyrroline (2AP), 2-(1-ethoxyethenyl)-1-pyrroline and 2-acetyl-1,4,5,6-tetrahydropyridine. “Fragrance” may also be produced when expression of a functional protein from the BADH homolog BADH2 is reduced or eliminated as described in International Publication No. 2006/032102 in the name of Grain Foods CRC Ltd, which is incorporated herein by reference.

It will be appreciated that a plant may produce fragrance only under certain conditions such as temperature, growth medium and light, ectopic application of chemicals etc. Fragrance may also be produced in one or more parts of the plant (as hereinbefore described), or throughout the entire plant. It is envisaged that aroma may result from secretion of one or more fragrant molecules from parts of the fragrant plant.

By “fragrant plant” is meant a plant, and in particular a crop plant, which has fragrance when compared to a non-fragrant plant. It will be appreciated by a skilled addressee that non-fragrant plants may produce one or more fragrance molecules but not to the threshold level which can be detected, particularly by human senses. It is envisaged that in preferred embodiments, fragrant plants are selected from the group consisting of rice, wheat, barley, oat, sorghum and Zea mays. Examples office varieties which are fragrant include jasmine, Basmati 370, Kyeema, Dellmont, Dragon's Eyeball 100, Dumsorhk, Goolarah, Gobindobhog and KDML, although without limitation thereto.

It will be appreciated by the skilled addressee that according to the present invention, fragrance may be detected, determined, measured or otherwise assayed, by one or a combination of suitable methods. It will be appreciated that such methods can be performed on samples obtained from a plant or a plant part, or alternatively can be performed in planta.

Sensory tests such as smell and taste, as are known in the art, may be employed to determine whether a plant has or retains at least a portion of a fragrance. By way of example, fragrance can be detected by tasting the associated flavour in individual seeds or assessing the aroma of leaf tissue or grains after either heating in water or reacting with solutions of KOH or I2-KI.

In certain embodiments which contemplate measurement or determination of a fragrance of a plant, one or more fragrance molecules in a plant or a plant part can be assayed by physicochemical assessment methods such as, liquid chromatography-mass spectrometry and gas chromatography-mass spectrometry (GC-MS) such as described in International Publication No. WO 2006/032102, which provides a non-limiting example of detecting 2-AP by GC-MS. Such methods are particularly useful when specific and sensitive detection methods are required.

In other particular embodiments, fragrance can be measured by a variety of nucleic acid techniques as are known in the art, to determine whether a plant has a fragrance genotype. In principle, any nucleic acid sequence detection technique may be applicable, such as nucleic acid sequencing, northern hybridization, southern hybridisation, nucleic acid sequence amplification, nucleic acid arrays and methods that detect melting temperature differences to identify whether a nucleic acid harbours one or more mutations and/or polymorphisms compared to a “wild type” reference nucleic acid.

In general embodiments, fragrance can be determined the presence or absence of a gene associated with or linked to fragrance, or alternatively detection of fragrance-associated mutations or polymorphisms in nucleic acid samples obtained from a plant. The invention also contemplates detection of variants, homologs and orthologs of a gene associated with or linked to fragrance. International Publication No. WO 2006/032102 describes the BADH2 allele which is truncated in fragrant varieties and has been linked to fragrance in fragrance varieties.

In preferred embodiments, the fragrance genotype is determined by using methods which detect a deletion within the BADH2 gene, whereby the deletion results in premature truncation of the BADH2 protein. Reference is made to International Publication No. WO 2006/032102, which describes use of a PCR based assay to detect a truncation in BADH2 which is associated with fragrance and is incorporated herein by reference.

The fragrance genotype can also be ascertained by sequencing of a nucleic acid, and in particular genomic DNA, to determine the presence or absence of a gene, allele, polymorphism or a mutation associated with or linked to fragrance.

It is also contemplated that methods described hereinbelow for detection of mutations or polymorphisms generally may be also applied to detecting fragrance-associated mutations and/or polymorphisms.

The term “nucleic acid” as used herein broadly designates single or double stranded mRNA, RNA, cRNA and DNA, said DNA inclusive of cDNA and genomic DNA. A nucleic acid may be native or recombinant and may comprise one or more artificial nucleotides, e.g. nucleotides not normally found in nature. RNA includes single-stranded and double-stranded unprocessed RNA, mRNA, siRNA, miRNA, RNAi and tRNA. Nucleic acid also encompasses modified purines (for example, inosine, methylinosine and methyladenosine) and modified pyrimidines (thiouridine and methylcytosine).

A “polynucleotide” is a nucleic acid having eighty (80) or more contiguous nucleotides, while an “oligonucleotide” has less than eighty (80) contiguous nucleotides.

By “genetic locus or loci” is meant the position of a gene in a linkage map or on a chromosome.

The term “gene” is used herein to describe a discrete nucleic acid locus, unit or region within a genome that may comprise one or more of introns, exons, splice sites, open reading frames and 5′ and/or 3′ non-coding regulatory sequences such as a a polyadenylation sequence.

Alternatively, protein-based techniques to detect a protein which is associated with or linked to fragrance may be applicable. Suitable methods include antibody-based methods such as, but not limited to immunoblot, immunoprecipitation and enzyme linked immunosorbent assay, mass spectrometry, two-dimensional electrophoresis, autoradiography and fluorescence spectroscopy.

By “protein” is meant an amino acid polymer. The amino acids may be natural or non-natural amino acids, D- or L-amino acids or chemically-derivatized amino acids as are well understood in the art.

A “peptide” is a protein having less than fifty (50) amino acids.

A “polypeptide” is a protein having fifty (50) or more amino acids.

Proteins and peptides may be useful in native, chemical synthetic or recombinant synthetic form.

It is also anticipated that function-based assays in which a protein is assayed for either a loss of function or gain of function, can also be utilised to detect fragrance. In preferred embodiments that relate to BAHD2, determination of the presence fragrance may be assayed by determining the affinity of the BADH2 protein (or a variant, homolog or ortholog thereof) for aminoaldehydes such as betaine aldehyde, γ-aminobutyraldehyde and γ-guanidinobutyraldehyde through monitoring the conversion of NAD+ to NADH by an increase in absorbance at 340 nm. Although not wishing to be bound by any particular theory, the presence a premature stop codon in a BADH2 allele codes for a substantially truncated BADH2 enzyme which, in turn, leads to inactivation of aminoaldehyde dehydrogenase activity. Bradbury et al, 2008, Plant Molecular Biology 68: 439-449 provides a non-limiting example of an enzymatic assay to determine the function of BADH2, and is incorporated herein by reference.

The present invention also contemplates use of fragments of amino acid sequences and/or nucleic acid sequences. In one embodiment, a “fragment” constitutes less than 100%, but at least 20%, preferably at least 30%, more preferably at least 80% or even more preferably at least 90% of a protein and/or nucleic acid sequence of interest.

Accordingly, in a particularly preferred aspect, the invention provides a method of producing a fragrant plant with improved crop performance, said method including the steps of propagating a fragrant plant with improved crop performance from a fragrant plant and a plant which has, or has been selected for, at least partial tolerance to one or more abiotic stress factors, to thereby produce a fragrant plant with improved crop performance.

In preferred embodiments, the fragrant plant does not have tolerance, or at least partial tolerance, to one or more abiotic stress factors.

In general embodiments, the step of propagating is inclusive of breeding and/or crossing parent plants with desirable physiological traits.

Although not wishing to be bound by any particular theory, methods of this aspect generally utilise the natural variation that is already present in the plant germplasm to select for a plant with abiotic stress tolerance and combining this trait with fragrance in order to create a fragrant plant with improved crop performance.

It will be appreciated that according to this aspect and all other aspects of the present invention, selecting or selection of a plant or plants can be from a population of fragrant plants, non-fragrant plants, or a combination thereof.

In the context of the present invention, by “abiotic stress”, “abiotic stresses” or “abiotic stress factors” is a meant one or a plurality of non-living factors or abnormal environmental parameters which have a negative impact on a living organism, and in particular a plant. Non-limiting examples of abiotic stresses (or otherwise known as abiotic stress factors) include drought, salinity, cold, freezing, high temperature, anoxia, high light intensity, acidity, nutrient imbalance and heavy metal tolerance. It will be appreciated that abiotic stresses in a plant can cause drastic yield reduction in most crops and this may be particularly acute for fragrant plant varieties. Typically, although not exclusively, abiotic stresses can result in dehydration or osmotic stress through reduced availability of water for vital cellular functions and maintenance of turgor pressure.

By “at least partially tolerant to one or more abiotic stress factors” or “at least partial tolerance to one or more abiotic stress factors” is meant that a plant (or part thereof), inclusive of a fragrant plant, is able to survive, resist or otherwise withstand exposure or treatment with one or more abiotic stress factors, or a level of one or more abiotic stress factors that would otherwise be detrimental to the plant (or part thereof). The dosage and/or time period (as is applicable) of the one or more abiotic stress factors is dependent on the plant and/or the abiotic stress factor that is used and can be readily determined by a person skilled in the art.

It is envisaged that selecting for a plant which is at least partially tolerant to one or more abiotic stress factors may include exposing or treating a plant to one or more abiotic stress factors in order to determine whether the plant is at least partially tolerant to the one or more abiotic stress factors.

In general, physiological traits such as tolerance to salt or other abiotic stress factors may be selected either directly or through the use of genetic and/or molecular biology techniques. Direct selection methods include exposing a plant or a part thereof, to one or more abiotic stress factors at a dosage and/or time period that is appropriate to determine which the plant or plants can survive under these conditions.

Genetic and/or molecular biology techniques as described herein are also applicable for the selection of a plant, or part thereof, with abiotic stress tolerance.

In light of the foregoing, it will be appreciated by a skilled addressee that the invention encompasses production of a fragrant plant with improved crop performance through conventional plant breeding techniques as are known in the art.

By “conventional plant breeding” is meant the creation of a new plant variety by hybridisation of two donor plants, one of which carries the trait of interest, followed by screening and field selection. This process is not reliant upon insertion or introduction of recombinant DNA in order to express a desired trait.

It will be appreciated by a person of skill in the art that a method for conventional plant breeding typically may comprise selecting a plant which is at least partially tolerant to one or more abiotic stress factors and propagating a fragrant plant from the plant which is at least partially tolerant to one or more abiotic stress factors to thereby produce a fragrant plant with improved crop performance. By way of example only, conventional plant breeding methods may include the following steps:

(a) identifying or selecting a first parent plant and a second parent plant, wherein the first parent plant is at least partially tolerant to one or more abiotic stress factors and the second parent plant has at least a portion of fragrance, and wherein the first and second plants are capable of cross-pollination. Screening methods inclusive of genetic screening methods and direct screening methods, or other screening methods as are well known to a person of skill in the art, and as are hereinbefore described, may be used to identify appropriate parents;

(b) pollinating the first parent plant with pollen from the second parent plant, or pollinating the second parent plant with pollen from the first parent plant;

(c) culturing the pollinated plant under conditions to produce progeny plants;

(d) selecting progeny plants that are homozygous or heterozygous for the quality trait using methods which are well known in the art.

It will be appreciated by those skilled in the art that once plants have been obtained which are heterozygous or homozygous for improved crop performance, those heterozygous or homozygous plants may be used in breeding programmes to transfer the ability for improved crop performance to fragrant plant varieties which lack improved crop performance.

In other particular aspects, the present invention also contemplates methods of producing a fragrant plant with improved crop performance, which includes the steps of introducing one or more mutations into genetic material of a fragrant plant and selecting a fragrant plant having one or more mutations, which is at least partially tolerant to one or more abiotic stress factors. According to these aspects, introduction of mutations potentially increases the ability of the fragrant plant to tolerate abiotic stress factors and result in improved crop performance.

The terms “mutant”,“mutation” and “mutated” are used herein generally to encompass conservative or non-conservative nucleic acid base pair substitutions, deletions and/or insertions introduced into a fragrant plant. In particular embodiments, the mutations are introduced into the genetic material of a fragrant plant. For example, mutations may be introduced into chromosomal DNA and genomic DNA, RNA such as unspliced and spliced mRNA, tRNA and other forms of genetic material as are known in the art.

The term “genetic material” is used herein to refer to one or more nucleic acid molecules which exist in a plant, in particularly in the germplasm of a plant. Non-limiting examples include genomic DNA, chromosomal DNA and RNA such as mRNA. Therefore, mutagenesis of the genetic material of a fragrant plant may result in introduction of mutations in one or a plurality of nucleic acid molecules.

In light of the foregoing, it will be appreciated that genome-wide mutagenesis of a fragrant plant is also contemplated. In alternative embodiments, mutations can also be introduced or induced by targeting specific loci or regions. It is envisaged that gain-of-function and loss-of-function mutations are contemplated as a result of the mutagenesis of the present invention, although without limitation thereto.

Mutations may be induced or introduced using either non-specific methods such as random mutagenesis or alternatively by using specific methods such as targeted mutagenesis. Contemplated by all methods are induction of single- or multiple-nucleotide substitutions, deletions and/or insertions, either alone or in combination. Mutagenesis methods of the present invention are inclusive of in vitro, in vivo and in planta methodology.

Chemical mutagenesis is a useful method of genome-wide random mutagenesis methods using alkylating agents such as ethylmethanesulfonate (EMS) and dimethyl sulfate (DMS) or other chemical mutagens such as ethidium bromide, formic acid, hyrdazine, sodium bisulphite and diepoxybutane.

Physical mutagenesis using physical mutagens as for example irradiation using ionising radiation (such as β, γ or X-ray radiation), UV irradiation and fast neutron irradiation of seeds may also be used for genome-wide random mutagenesis. It will be appreciated by a person skilled in the art that the time and dosage of exposure of the plant or a part thereof, to a mutagen is dependent on the plant and mutagen that is used and can be readily determined by a skilled addressee.

Mutations may be introduced into nucleic acids by random or site-directed mutagenesis as are well known in the art. Non-limiting examples of nucleic acid mutagenesis methods are provided in Chapter 8 of CURRENT PROTOCOLS IN MOLECULAR BIOLOGY Eds Ausubel et al., (John Wiley & Sons, Inc. 1995-2008) and is incorporated herein by reference.

Random mutagenesis methods also include incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein. It is also noted that PCR-based random mutagenesis kits are commercially available, such as the Diversify™ kit (Clontech).

In certain embodiments, mutations are introduced into the genetic material of a fragrant plant by a nucleic acid sequence amplification-based technique.

As used herein, a “nucleic acid sequence amplification technique” includes but is not limited to polymerase chain reaction (PCR) as for example described in Chapter 15 of CURRENT PROTOCOLS IN MOLECULAR BIOLOGY, Eds. Ausubel et al. (John Wiley & Sons NY USA 1995-2001) strand displacement amplification (SDA); rolling circle replication (RCR) as for example described in International Application WO 92/01813 and International Application WO 97/19193; nucleic acid sequence-based amplification (NASBA) as for example described by Sooknanan et al. 1994, Biotechniques 17 1077; ligase chain reaction (LCR) as for example described in International Application WO89/09385 and Chapter 15 of CURRENT PROTOCOLS IN MOLECULAR BIOLOGY supra; Q-β replicase amplification as for example described by Tyagi et al. 1996, Proc. Natl. Acad. Sci. USA 93: 5395 and helicase-dependent amplification as for example described in International Publication WO 2004/02025.

Region-specific mutagenesis and directed mutagenesis using PCR may also be employed to construct nucleic acid mutants according to the invention. Oligonucleotide-mediated (or site-directed) mutagenesis may also be used. A non-limiting example of oligonucleotide-mediated site-directed mutagenesis procedures to introduce small clusters of point mutations throughout the target region is provided in Ausubel et al., supra. Briefly, mutations are introduced into a sequence by annealing a synthetic oligonucleotide containing one or more mismatches to the sequence of interest cloned into a single-stranded M13 vector. This template is grown in an Escherichia coli dut⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA. It is also noted that site-directed mutagenesis kits are commercially available, such as the QuikChange™ kit (Stratagene).

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8, incorporated herein by reference) which describes a first protocol that uses complementary oligonucleotides and requires a unique restriction site adjacent to the region that is to be mutagenised. A nested series of deletion mutations is first generated in the region. A pair of complementary oligonucleotides is synthesised to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series.

Mutations may be induced or introduced by insertion of one or a plurality of nucleotides or base-pairs into the genetic material. Transposon and retrotransposon mutagenesis (for example as described in Walbot, Curr Opin Plant Biol. 2000 April; 3(2):103-7; U.S. Pat. No. 6,720,479; Voytas (1996) Genetics 142:569-578) are also contemplated as methods for insertional mutagenesis. Other methods of insertional mutagenesis include targeted methods such as homologous recombination and site-specific recombination. A non-limiting example of homologous recombination is the T-DNA system (for example as described in Wang et al, 2001, Gene, 272: 249-255; and Iida and Terada, 2005, Plant Molecular Biology, 59: 205-219). An example of site-specific recombination is the cre-lox recombination system of bacteriophage P1, which has been applied to promote recombination of specific locations on the genome of plants cells (for example, as described in U.S. Pat. No. 5,658,772).

Chimeric RNA/DNA oligonucleotide-directed gene targeting is also a useful technique for the generation of site-specific point mutations such as deletions, insertions and/or base changes in higher plants (see for example as described in Iida and Terada, 2005, Plant Molecular Biology, 59: 205-219; and Rice et al, 2000, Plant Physiol, 123: 427-43 8).

Mutations may also be introduced by deletional mutagenesis of one or a plurality of nucleotides, or a region of a genetic loci. For example, fast neutron deletion mutagenesis is contemplated by the present invention as a genome-wide deletional mutagenesis method and utilises fast neutron bombardment to create a random mutagenised populations of plants, and more particularly knockout mutations such as described Li et al. Comp Funct Genomics, (2002) 3:158-60. It will be appreciated that targeted deletional mutagenesis may be achieved by using a variety of other nucleic acid based mutagenesis methods as herein described, such as, but not limited to oligonucleotide-based mutagenesis.

It is envisaged that Targeting Induced Local Lesions in Genomes (otherwise referred to as “TILLING”) is particularly amenable for random mutagenesis to generate point mutations in plants. TILLING combines traditional chemical mutagenesis following by high-throughput screening for point mutations. Reference is made to McCallum et al., Nat. Biotechnol. (2000) 18, 455-457; Till et al., Methods Mol. Biol. (2003) 236: 205-220; Henikoff et al, (2004) Plant Physiology 135: 630-636 and Till et al. Genome Res. (2003) 13: 524-530 for non-limiting examples of TILLING methods applicable to the present invention. TILLING is also particularly amenable to high-throughput methodology, as described in Henikoff et al (2003) Annual Rev Plant Biology, 54: 375-401, which provides non-limiting examples of high-throughput TILLING methods, technologies for the detecting of single-nucleotide differences and TILLING generally and is incorporated herein by reference.

A person of skill in the art will readily appreciate that post-transcriptional gene silencing is also an attractive method of targeting specific loci in a plant.

Silencing can be achieved by introduction of synthetic recombinant molecules or transgenes targeted to disrupt or degrade specific nucleotide sequences. Hence according to one embodiment, silencing can occur by construction of a knockout gene. Typically, although not exclusively, a gene knockout is created by homologous recombination of a foreign sequence into the gene of interest, to thereby disrupt the gene.

According to another embodiment, silencing may involve generation of an inhibitory RNA molecule (hereinafter referred to as “RNAi”). RNAi, and in particular siRNA (but not limited thereto), involves sequence specific cleavage of a cognate mRNA. Therefore the present invention contemplates generation of genetic reagents for RNAi wherein the genetic reagents comprises one or more nucleotide sequences capable of directing synthesis of an RNA molecule, said nucleotide sequence selected from the list comprising:—

(i) a nucleotide sequence transcribable to an RNA molecule comprising an RNA sequence which is substantially homologous to an RNA sequence encoded by a nucleotide sequence within the genome of said pathogen;

(ii) a reverse complement of the nucleotide sequence of (i);

(iii) a combination of the nucleotide sequences of (i) and (ii),

(iv) multiple copies of nucleotide sequences of (i), (ii) or (iii), optionally separated by a spacer sequence;

(v) a combination of the nucleotide sequences of (i) and (ii), wherein the nucleotide sequence of (ii) represents an inverted repeat of the nucleotide sequence of (i), separated by a spacer sequence; and

(vi) a combination as described in (v), wherein the spacer sequence comprises an intron sequence spliceable from said combination;

Where the nucleotide sequence comprises an inverted repeat separated by a non-intron spacer sequence, upon transcription, the presence of the non-intron spacer sequence facilitates the formation of a stem-loop structure by virtue of the binding of the inverted repeat sequences to each other. The presence of the non-intron spacer sequence causes the transcribed RNA sequence (also referred to herein as a “transcript”) so formed to remain substantially in one piece, in a form that may be referred to herein as a “hairpin”. Alternatively, where the nucleotide sequence comprises an inverted repeat wherein the spacer sequence comprises an intron sequence, upon transcription, the presence of intron/exon splice junction sequences on either side of the intron sequence facilitates the removal of what would otherwise form into a loop structure. The resulting transcript comprises a double-stranded RNA (dsRNA) molecule, optionally with overhanging 3′ sequences at one or both ends. Such a dsRNA transcript is referred to herein as a “perfect hairpin”. The RNA molecules may comprise a single hairpin or multiple hairpins including “bulges” of single-stranded DNA occurring in regions of double-stranded DNA sequences.

It will be appreciated that the methods of the present invention to produce a fragrant plant with improved crop performance by introducing or inducing one or a plurality of mutations includes step of determining whether the mutated fragrant plant is at least partially tolerant to one or more abiotic stress factors by using selection or screening methods such as exposing the mutant fragrant plant to one or a variety of abiotic stresses as hereinbefore described. It is also contemplated that selection or screening using specific DNA markers may also be employed using nucleic acid techniques as described herein.

The present invention also contemplates methods to detect the mutation or polymorphism arising from the mutagenesis as herein described.

Typically, although not exclusively, nucleotide sequence polymorphisms may be identified by nucleotide sequencing as is well known in the art. Extensive methodology relating to nucleotide sequencing is provided in Chapter 7 of CURRENT PROTOCOLS IN MOLECULAR BIOLOGY, Eds. Ausubel et al. John Wiley & Sons NY USA (1995-2002).

In general embodiments, a nucleic acid sequence amplification technique may be useful for rapid detection of a genetic loci, mutation or a nucleotide sequence polymorphism, particularly where multiple samples are to be tested.

As used herein, a “nucleic acid sequence amplification technique” includes but is not limited to polymerase chain reaction (PCR) as for example described in Chapter 15 of CURRENT PROTOCOLS IN MOLECULAR BIOLOGY Eds. Ausubel et al. (John Wiley & Sons NY USA 1995-2001) strand displacement amplification (SDA); rolling circle replication (RCR) as for example described in International Application WO 92/01813 and International Application WO 97/19193; nucleic acid sequence-based amplification (NASBA) as for example described by Sooknanan et al. 1994, Biotechniques 17 1077; ligase chain reaction (LCR) as for example described in International Application WO89/09385 and Chapter 15 of CURRENT PROTOCOLS IN MOLECULAR BIOLOGY supra; Q-β replicase amplification as for example described by Tyagi et al. 1996, Proc. Natl. Acad. Sci. USA 93 5395 and helicase-dependent amplification as for example described in International Publication WO 2004/02025.

In this regard, it will be appreciated than an RNA copy of DNA corresponds to the DNA notwithstanding the presence of uracil bases rather than thymine bases.

In particular, multiplex PCR (optimally fluorescence multiplex PCR) may be advantageous for detection of fragrance-associated or abiotic stress-associated genetic markers, in addition to polymorphisms, particularly for high-throughput genetic analysis.

Another potentially useful PCR method may be Bi-PASA (Bidirectional PCR Amplification of Specific Alleles), as for example described in Liu et al. 1997, Genome Res. 7 389-399.

Yet another potentially useful PCR method is allele-specific oligonucleotide hybridization, as for example described in Aitken et al., 1999, J. Natl. Cancer Inst 91 446-452; Kwok, 2000, Pharmacogenomics 1 95; and Shattuck-Eidens et al, 1991, 8:240-5.

It will also be well understood by the skilled person that detection of nucleotide sequence polymorphisms may be performed using any of a variety of techniques such as PCR-RFLP analysis, RFLP analysis fluorescence-based melt curve analysis, SSCP analysis, denaturing gradient gel electrophoresis (DGGE), heteroduplex analysis, denaturing high performance liquid chromatography (DHPLC) or direct sequencing of amplification products.

Melt curve analysis can be performed using fluorochrome-labeled allele-specific probes which form base-pair mismatches when annealing to wild-type DNA strands in heterozygotes. Alternatively, fluorescent DNA-intercalating dyes such as SYBR Green 1 can reveal the presence of these base-pair mismatches by virtue of their lower melting temperature (Tₘ) compared to fully complementary sequences. A useful example of allele-specific melt curve analysis can be found, for example, in International Publication No. WO97/46714 and International Publication No WO02/30946.

DGGE also exploits Tₘ differences, but uses differential electrophoretic migration through gradient gels as a means of distinguishing subtle nucleotide sequence differences between alleles. Examples of DGGE methods can be found in Fodde & Losekoot, 1994, Hum. Mutat. 3 83-9 and U.S. Pat. Nos. 5,045,450 and 5,190,856.

SSCP detection may be performed using gel based systems such as described in Nataraj et al., 1999, Electrophoresis 20 1177 or by capillary electrophoresis such as described in Ren, 2000, J. Chromatogr. B. Biomed. Sci. Appl. 12 741, the latter being particularly suited to high-throughput genetic analysis.

Heteroduplex analysis in which mismatches in DNA heteroduplexes formed between a reference DNA and a test DNA (typically amplified by PCR) indicate the site of a mutation or polymorphism, that are characterised by sequencing, may also be undertaken to detect a single-nucleotide polymorphism. Henikoff et al (2003) Annual Rev Plant Biology, 54: 375-401 provides non-limiting examples of suitable methods of heteroduplex analysis and is incorporated herein by reference. Heteroduplex analysis is readily application to a large number of samples, in particular pooled DNA and moreover, can be adapted so that the final analysis is performed by chromatography, miniature electrophoresis on a ‘chip’, and acrylamide gel electrophoresis.

DHPLC is applicable for detection of single-nucleotide polymorphisms as well as small deletions and insertions such as described in Sivakumaran et al, 2003, CURRENT SCIENCE, VOL. 84, page 291. DHPLC detects mutations on the basis of mismatches between amplified chromosomal fragments that result in the formation of heteroduplices. The heteroduplices, which are thermally less stable than their corresponding homoduplices, are resolved by means of ionpair reversed-phase liquid chromatography at elevated column temperatures typically in the range of 50-70° C. depending on the GC-content of the sequences.

With regard to high-throughput genetic analysis, the invention contemplates methods that utilize nucleic acid arrays.

In one embodiment, a library or array comprising one or more fragrance-associated nucleic acids may be used to screen fragrant plant samples, particularly with regard to high-throughput genetic analysis. In other embodiments, a library or array may comprise one or more nucleic acids associated with or linked to one or more abiotic stress factors to screen for plant samples.

In one particular form of this embodiment, the invention provides a molecular library in the form of a nucleic acid array that comprises a substrate to which is immobilized, bound or otherwise coupled an fragrance-associated nucleic acid or a nucleic acid associated with or linked to one or more abiotic stress factors, or a fragment thereof. Each immobilized, bound or otherwise coupled nucleic acid has an “address” on the array that signifies the location and identity of said nucleic acid.

Nucleic acid array technology has become well known in the art and examples of methods applicable to array technology are provided in Chapter 22 of CURRENT PROTOCOLS IN MOLECULAR BIOLOGY, Eds. Ausubel et al. (John Wiley & Sons NY USA 1995-2001).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

With regard to the above, nucleic acid samples for genetic analysis may be isolated from any cell or tissue source, inclusive of endosperm tissue. For example such tissues may include but are not restricted to leaves, roots, stems and seeds.

In other general embodiments, the methods of the invention may involve measuring expression levels of nucleic acids associated with or linked to fragrance and/or abiotic stress tolerance, compared to a reference sample.

Methods for quantification for nucleic acids are well known in the art. Measurement of relative amounts of nucleic acid levels associated with or linked to fragrance and/or abiotic stress tolerance compared to an expressed level of a reference nucleic acid may be conveniently performed using a nucleic acid array as hereinbefore described. Alternative methods include hybridisation techniques such as northern hybridisation, as are well known in the art.

In another particular form of this embodiment, quantitative or semi-quantitative PCR using primers corresponding to nucleic acids of interest may be used to quantify relative expression levels of the nucleic acid to thereby determine whether a plant retains at least a portion of fragrance or whether a plant is tolerant to one or more abiotic stress factors. In particularly preferred embodiments, quantitative reverse transcriptase PCR may be used to quantify cDNA in a nucleic acid sample, which thereby gives a measure of mRNA transcript levels.

PCR amplification is not linear and hence end point analysis does not always allow for the accurate determination of nucleic acid expression levels. Real-time PCR analysis provides a high throughput means of measuring gene expression levels. It uses specific primers, an intercalating fluorescent dye such as SYBR Green I or ethidium bromide (EtBr) and fluorescence detection to measure the amount of product after each cycle. Hybridization probes utilise either quencher dyes or fluorescence directly to generate a signal. This method may be used to validate and quantify nucleic acid expression differences in cells or tissues obtained from a fragrant plant compared to cells or tissues obtained from a fragrant plant with improved crop performance, for example.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably comprising 15-60 contiguous nucleotides, which is capable of annealing to a complementary nucleic acid “template” and being extended in a template-dependent fashion by the action of a DNA polymerase such as Tag polymerase, RNA-dependent DNA polymerase or Sequenase™.

According to these aspects, the step of determining whether the fragrant plant having one or more mutations is at least partially tolerant to one or more abiotic stress factors includes selection methods as herein described, for example by exposure to one or more abiotic stress factors or by genetic screening, although without limitation thereto.

In light of the foregoing, it will be appreciated by a skilled addressee that the invention encompasses production of a fragrant plant with improved crop performance through recombinant DNA methodology.

That is, in other general aspects, the invention provides a method of producing a fragrant plant with improved crop performance wherein the method includes the step of genetically modifying one or more fragrant plant cells or tissues to thereby produce a genetically-modified fragrant plant with improved crop performance, wherein said genetically-modified fragrant plant with improved crop performance retains at least a portion of a fragrance. In preferred embodiments, the aforementioned genetic modification confers at least partial tolerance to one or more abiotic stress factors.

The term “genetically-modified” or “genetic modification” broadly refers to introduction of a heterologous nucleic acid into a plant. The heterologous nucleic acid may subsist in the organism by means of chromosomal integration into the host genome or alternatively, by episomal replication. “Genetic modification” or “genetically-modified” includes within its scope “transgenic” plants, as typically used in the art.

Preferably, genetic-modification results in a fragrant plant with improved crop performance whilst retaining at least a portion of fragrance.

By “retain”, “retains”, “retained” or “retaining” is meant to keep, preserve, save or otherwise maintain at least a portion, amount or level of fragrance in a genetically-modified fragrant plant. Preferably, the introduction of the heterologous nucleic acid into a fragrant plant does not perturb, substantially disrupt, or substantially diminish fragrance in the fragrant plant.

In particularly preferred embodiments of this aspect, the method further includes the step of determining whether genetically modified fragrant plant retains at least a portion of fragrance, using methods as herein described.

According to aspects of the present invention which contemplate methods of producing a genetically-modified plant, at least a portion of a fragrance is retained by ensuring that the biochemical pathway which leads to fragrance remains unperturbed or undisturbed upon introduction of the heterologous nucleic acid into a plant.

In preferred embodiments, the heterologous nucleic acid confers at least partial tolerance to one or more abiotic stress factors. Non-limiting examples of suitable heterologous nucleic acids include cDNAs or nucleotide sequences encoding proteins such as tomato ethylene response factor (TERF1 for example as described in Gao et al, 2008, Plant Cell Rep), LeNHX2 (see, for example, Rodriguez-Rosales et al, 2008, New Phytologist, 179: 366-377), SDIR1 (for example, Zhang et al, Biosci Biotechnol Biochem, 72: 2251-2254), chloroplastic glutamine synthetase (GS2; as for example in Hoshida et al, Plant Molecular Biology, 2000, 43: 102-111), HVA1 (as for example in Xu et al, 1996, Plant Physiology, 110: 249-257), the vacuolar Na⁺/H⁺ antiporter from Pennisetum glaucum (PgNXH1; as for example in Verma et al, 2007, J Biosci, 32: 621-628), stress responsive gene SNAC1 (see for example Hu et al, 2006, PNAS, 103: 12987-12992) and a fusion of the trehalose-6-phosphate synthase and trehalose-6-phosphate phosphatase (as for example described in Jang et al, 2003, Plant Physiology, 131: 516-524).

Suitably, production of genetically-modified plants of the invention includes the steps of:

(i) introducing one or more genetic constructs to a fragrant plant cell or tissue; and

(ii) selectively propagating a genetically-modified fragrant plant from the plant cell or tissue in (i), wherein the genetically-modified fragrant plant retains at least a portion of fragrance.

A “genetic construct” comprises a heterologous nucleic acid encoding one or more proteins which confer at least partial tolerance to one or more abiotic stress factors and one or more additional nucleotide sequences that facilitate manipulation, propagation and/or expression of the heterologous nucleic acid.

It can be readily appreciated by a person skilled in the art that a genetic construct is a nucleic acid comprising any one of a number of nucleotide sequence elements, the function of which depends upon the desired use of the construct. Uses range from vectors for the general manipulation and propagation of recombinant DNA to more complicated applications such as prokaryotic or eukaryotic expression of a heterologous nucleic acid and production of genetically-modified plants. Typically, although not exclusively, genetic constructs are designed to provide more than one application. By way of example only, a genetic construct whose intended end use is recombinant protein expression in a eukaryotic system may have incorporated nucleotide sequences for such functions as cloning and propagation in prokaryotes over and above sequences required for expression. An important consideration when designing and preparing such genetic constructs are the required nucleotide sequences for the intended application.

In view of the foregoing, it is evident to a person of skill in the art that genetic constructs are versatile tools that can be adapted for any one of a number of purposes.

By “vector” is meant a nucleic acid, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integratable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

In a preferred embodiment, the genetic construct is an expression construct suitable for genetic modification of plants, wherein the isolated nucleic acid is operably linked or connected to one or more regulatory sequences in an expression vector.

An “expression vector” may be either a self-replicating extra-chromosomal or episomal vector such as a plasmid, or a vector that integrates into a host genome.

By “operably linked or connected” is meant that said one or more regulatory nucleotide sequence(s) is/are positioned relative to the nucleic acid encoding the protein to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

The genetic constructs of the present invention can further include enhancers, either translation or transcription enhancers, as may be required. These enhancer regions are well known to persons skilled in the art, and can include the ATG initiation codon and adjacent sequences. The initiation codon must be in phase with the reading frame of the coding sequence relating to the heterologous or endogenous DNA sequence to ensure translation of the entire sequence. The translation control signals and initiation codons can be of a variety of origins, both natural and synthetic. Translational initiation regions may be provided from the source of the transcriptional initiation region, or from the heterologous or endogenous DNA sequence. The sequence can also be derived from the source of the promoter selected to drive transcription, and can be specifically modified so as to increase translation of the mRNA.

Examples of transcriptional enhancers include, but are not restricted to, elements from the CaMV 35S promoter and octopine synthase genes as for example described by Last et al. (U.S. Pat. No. 5,290,924, which is incorporated herein by reference). It is proposed that the use of an enhancer element such as the ocs element, and particularly multiple copies of the element, will act to increase the level of transcription from adjacent promoters when applied in the context of plant transformation.

As the DNA sequence inserted between the transcription initiation site and the start of the coding sequence, i.e., the untranslated leader sequence, can influence gene expression, one can also employ a particular leader sequence. Preferred leader sequences include those that comprise sequences selected to direct optimum expression of the heterologous or endogenous DNA sequence. For example, such leader sequences include a preferred consensus sequence which can increase or maintain mRNA stability and prevent inappropriate initiation of translation as for example described by Joshi (1987, Nucl. Acid Res., 15:6643), which is incorporated herein by reference. However, other leader sequences, e.g., the leader sequence of RTBV, have a high degree of secondary structure that is expected to decrease mRNA stability and/or decrease translation of the mRNA. Thus, leader sequences (i) that do not have a high degree of secondary structure, (ii) that have a high degree of secondary structure where the secondary structure does not inhibit mRNA stability and/or decrease translation, or (iii) that are derived from genes that are highly expressed in plants, will be most preferred.

Regulatory elements such as the sucrose synthase intron as, for example, described by Vasil et al. (1989, Plant Physiol., 91:5175), the Adh intron I as, for example, described by Callis et al. (1987, Genes Develop., II), or the TMV omega element as, for example, described by Gallie et al. (1989, The Plant Cell, 1:301) can also be included where desired. Other such regulatory elements useful in the practice of the invention are known to those of skill in the art.

Additionally, targeting sequences may be employed to target a protein product of the heterologous or endogenous nucleotide sequence to an intracellular compartment within plant cells or to the extracellular environment. For example, a DNA sequence encoding a transit or signal peptide sequence may be operably linked to a sequence encoding a desired protein such that, when translated, the transit or signal peptide can transport the protein to a particular intracellular or extracellular destination, respectively, and can then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane. For example, the transit or signal peptide can direct a desired protein to a particular organelle such as a plastid (e.g., a chloroplast), rather than to the cytoplasm. Thus, the genetic construct can further comprise a plastid transit peptide encoding DNA sequence operably linked between a promoter region or promoter variant according to the invention and the heterologous or endogenous nucleotide sequence. For example, reference may be made to Heijne et al. (1989, Eur. J. Biochem., 180:535) and Keegstra et al. (1989, Ann. Rev. Plant Physiol. Plant Mol. Biol., 40:471), which are incorporated herein by reference.

An isolated nucleic acid of the present invention can also be introduced into a vector, such as a plasmid. Plasmid vectors include additional DNA sequences that provide for easy selection, amplification, and transformation of the expression cassette in prokaryotic and eukaryotic cells, e.g., pUC-derived vectors, pSK-derived vectors, pGEM-derived vectors, pSP-derived vectors, or pBS-derived vectors. Additional DNA sequences include origins of replication to provide for autonomous replication of the vector, selectable marker genes, preferably encoding antibiotic or herbicide resistance, unique multiple cloning sites providing for multiple sites to insert DNA sequences or genes encoded in the DNA construct, and sequences that enhance transformation of prokaryotic and eukaryotic cells.

The vector preferably contains an element(s) that permits stable integration of the vector into the host cell genome or autonomous replication of the vector in the cell independent of the genome of the cell. The vector may be integrated into the host cell genome when introduced into a host cell. For integration, the vector may rely on the heterologous or endogenous DNA sequence or any other element of the vector for stable integration of the vector into the genome by homologous recombination. Alternatively, the vector may contain additional nucleic acid sequences for directing integration by homologous recombination into the genome of the host cell. The additional nucleic acid sequences enable the vector to be integrated into the host cell genome at a precise location in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences.

For autonomous replication, the vector may further comprise an origin of replication enabling the vector to replicate autonomously in the host cell in question. Examples of bacterial origins of replication are the origins of replication of plasmids pBR322, pUC19, pACYC 177, and pACYC 184 permitting replication in E. coli, and pUB110, pE194, pTA1060, and pAM.beta.1 permitting replication in Bacillus. The origin of replication may be one having a mutation to make its function temperature-sensitive in a Bacillus cell (see, e.g., Ehrlich, 1978, Proc. Natl. Acad. Sci. USA 75:1433).

To facilitate identification of transformants, the genetic construct desirably comprises a selectable or screenable marker gene as, or in addition to, the expressible heterologous or endogenous nucleotide sequence. The actual choice of a marker is not crucial as long as it is functional (i.e., selective) in combination with the plant cells of choice. The marker gene and the heterologous or endogenous nucleotide sequence of interest do not have to be linked, since co-transformation of unlinked genes as, for example, described in U.S. Pat. No. 4,399,216 is also an efficient process in plant transformation.

Included within the terms selectable or screenable marker genes are genes that encode a “secretable marker” whose secretion can be detected as a means of identifying or selecting for transformed cells. Examples include markers that encode a secretable antigen that can be identified by antibody interaction, or secretable enzymes that can be detected by their catalytic activity. Secretable proteins include, but are not restricted to, proteins that are inserted or trapped in the cell wall (e.g., proteins that include a leader sequence such as that found in the expression unit of extensin or tobacco PR-S); small, diffusible proteins detectable, e.g. by ELISA; and small active enzymes detectable in extracellular solution (e.g., α-amylase, β-lactamase, phosphinothricin acetyltransferase).

Examples of bacterial selectable markers are the dal genes from Bacillus subtilis or Bacillus licheniformis, or markers that confer antibiotic resistance such as ampicillin, kanamycin, erythromycin, chloramphenicol or tetracycline resistance. Exemplary selectable markers for selection of plant transformants include, but are not limited to, a hyg gene which encodes hygromycin B resistance; a neomycin phosphotransferase (neo) gene conferring resistance to kanamycin, paromomycin, G418 and the like as, for example, described by Potrykus et al. (1985, Mol. Gen. Genet. 199:183); a glutathione-S-transferase gene from rat liver conferring resistance to glutathione derived herbicides as, for example, described in EP-A 256 223; a glutamine synthetase gene conferring, upon overexpression, resistance to glutamine synthetase inhibitors such as phosphinothricin as, for example, described WO87/05327, an acetyl transferase gene from Streptomyces viridochromogenes conferring resistance to the selective agent phosphinothricin as, for example, described in EP-A 275 957, a gene encoding a 5-enolshikimate-3-phosphate synthase (EPSPS) conferring tolerance to N-phosphonomethylglycine as, for example, described by Hinchee et al. (1988, Biotech., 6:915), a bar gene conferring resistance against bialaphos as, for example, described in WO91/02071; a nitrilase gene such as bxn from Klebsiella ozaenae which confers resistance to bromoxynil (Stalker et al., 1988, Science, 242:419); a dihydrofolate reductase (DHFR) gene conferring resistance to methotrexate (Thillet et al., 1988, J. Biol. Chem., 263:12500); a mutant acetolactate synthase gene (ALS), which confers resistance to imidazolinone, sulfonylurea or other ALS-inhibiting chemicals (EP-A-154 204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known; a β-galactosidase gene encoding an enzyme for which chromogenic substrates are known; an aequorin gene (Prasher et al., 1985, Biochem. Biophys. Res. Comm., 126:1259), which may be employed in calcium-sensitive bioluminescence detection; a green fluorescent protein gene (Niedz et al., 1995 Plant Cell Reports, 14:403); a luciferase (luc) gene (Ow et al., 1986, Science, 234:856), which allows for bioluminescence detection; a β-lactamase gene (Sutcliffe, 1978, Proc. Natl. Acad. Sci. USA 75:3737), which encodes an enzyme for which various chromogenic substrates are known (e.g., PADAC, a chromogenic cephalosporin); an R-locus gene, encoding a product that regulates the production of anthocyanin pigments (red color) in plant tissues (Dellaporta et al., 1988, in Chromosome Structure and Function, pp. 263-282); an α-amylase gene (Ikuta et al., 1990, Biotech., 8:241); a tyrosinase gene (Katz et al., 1983, J. Gen. Microbiol., 129:2703) which encodes an enzyme capable of oxidizing tyrosine to dopa and dopaquinone which in turn condenses to form the easily detectable compound melanin; or a xylE gene (Zukowsky et al., 1983, Proc. Natl. Acad. Sci. USA 80:1101), which encodes a catechol dioxygenase that can convert chromogenic catechols.

The initial step in production of a genetically-modified plant is introduction of DNA into a plant host cell. A number of techniques are available for the introduction of DNA into a plant host cell. There are many plant transformation techniques well known to workers in the art, and new techniques are continually becoming known. The particular choice of a transformation technology will be determined by its efficiency to transform certain plant species as well as the experience and preference of the person practising the invention with a particular methodology of choice. It will be apparent to the skilled person that the particular choice of a transformation system to introduce a genetic construct into plant cells is not essential to or a limitation of the invention, provided it achieves an acceptable level of nucleic acid transfer. Guidance in the practical implementation of transformation systems for plant improvement is provided by Birch (1997, Annu. Rev. Plant Physiol. Plant Molec. Biol. 48: 297-326), which is incorporated herein by reference.

In one embodiment, transformation is by microprojectile bombardment, for example as described by Franks & Birch, 1991, Aust. J. Plant. Physiol., 18:471; Gambley et al., 1994, supra; and Bower et al., 1996, Molecular Breeding, 2:239, which are herein incorporated by reference.

In another embodiment, transformation is Agrobacterium-mediated. Examples of Agrobacterium-mediated transformation of monocots are provided in U.S. Pat. No. 6,037,522, Hiei et al., 1994, Plant Journal 6 271 and Ishida et al., 1996, Nature Biotechnol. 14 745 in relation to various cereals, Arencibia et al., 1998, Transgenic Res. 7 213.

Accordingly, persons skilled in the art will be aware that a variety of other transformation methods are applicable to the method of the invention such as liposome-mediated (Ahokas et al., 1987, Heriditas 106 129), laser-mediated (Guo et al., 1995, Physiologia Plantarum 93 19), silicon carbide or tungsten whiskers (U.S. Pat. No. 5,302,523; Kaeppler et al., 1992, Theor. Appl. Genet. 84 560), virus-mediated (Brisson et al., 1987, Nature 310 511), polyethylene-glycol-mediated (Paszkowski et al., 1984, EMBO J. 3 2717) as well as transformation by microinjection (Neuhaus et al., 1987, Theor. Appl. Genet. 75 30) and electroporation of protoplasts (Fromm et al., 1986, Nature 319 791).

Alternatively, a combination of different techniques may be employed to enhance the efficiency of the transformation process, e.g., bombardment with Agrobacterium coated microparticles (EP-A-486234) or microprojectile bombardment to induce wounding followed by co-cultivation with Agrobacterium (EP-A-486233).

The methods used to regenerate transformed cells into differentiated plants are not critical to this invention, and any method suitable for a target plant can be employed. Normally, a plant cell is regenerated to obtain a whole plant following a transformation process.

The term “regeneration” as used herein means growing a whole, differentiated plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible. Regeneration also occurs from plant callus, explants, organs or parts. Shoots that develop are excised from calli and transplanted to appropriate root-inducing selective medium. Rooted plantlets are transplanted to soil as soon as possible after roots appear. The plantlets can be repotted as required, until reaching maturity.

For example, wheat plants have been regenerated from embryogenic suspension culture by selecting only the aged compact and nodular embryogenic callus tissues for the establishment of the embryogenic suspension cultures (Vasil, 1990, Bio/Technol. 8:429-434). The combination with transformation systems for these crops enables the application of the present invention to monocots.

In vegetatively propagated crops, the mature transgenic plants are propagated by the taking of cuttings or by tissue culture techniques to produce multiple identical plants. Selection of desirable transgenotes is made and new varieties are obtained and propagated vegetatively for commercial use.

In seed propagated crops, the mature transgenic plants can be self-crossed to produce a homozygous inbred plant. The inbred plant produces seed containing the newly introduced heterologous gene(s). These seeds can be grown to produce plants that would produce a selected phenotype, e.g., increased abiotic stress tolerance or fragrance.

Parts obtained from the regenerated plant, such as grains, cells, tissues, leaves, fruit, flowers, seeds and other reproductive material, material useful for vegetative propagation, F1 hybrids, male-sterile plants branches and the like are included in the invention, provided that these parts comprise cells that have been transformed as described. Include are all other plants and plant products derivable from a genetically-modified plant of the invention. Progeny and variants, and mutants of the regenerated plants are also included within the scope of the invention, provided that these parts comprise the introduced nucleic acid sequences.

It will be appreciated that the literature describes numerous techniques for regenerating specific plant types and more are continually becoming known. Those of ordinary skill in the art can refer to the literature for details and select suitable techniques without undue experimentation.

To confirm the presence of the heterologous nucleic acid in the regenerating plants, a variety of assays may be performed. Such assays include, for example, “molecular biological” assays well known to those of skill in the art, such as Southern and Northern blotting and PCR; a protein expressed by the heterologous DNA may be analysed by western blotting, high performance liquid chromatography or ELISA (e.g., nptII) as is well known in the art.

Examples of various methods applicable to characterization of transgenic plants are provided in Chapters 9 and 11 of PLANT MOLECULAR BIOLOGY: A Laboratory Manual Ed. M. S. Clark (Springer-Verlag, Heidelberg, 1997), which chapters are herein incorporated by reference.

In another aspect of the invention, the invention provides a genetically-modified fragrant plant with improved crop performance, wherein the a genetically-modified fragrant plant with improved crop performance is at least partially tolerant to one or more abiotic stress factors.

In particular embodiments, the heterologous nucleic acid is not codA, TERF1 or PgNXH1.

Also contemplated are cells, tissues, leaves, fruit, flowers, seeds and other reproductive material, material useful for vegetative propagation, F1 hybrids, male-sterile plants and all other plants and plant products derivable from said genetically-modified plant.

The plants identified or produced by the methods of the invention may be used to produce any food product for which that organism is suitable. For example, cereal crops may be used to produce rice, flour and grains for use in the production of food products such as, for example, bread, beer and other fermented and non-fermented beverages.

It will be appreciated that the present invention is applicable to any plant cell or tissue derivable from a plant or a part thereof, inclusive of meristem, leaf, axillary bud, stems, shoot apex, leaf sheath, petioles, root, inflorescence, leaf sheath, flower stalks, although without limitation thereto.

So that the invention may be readily understood and put into practical effect, reference is made to the following non-limiting Examples.

### EXAMPLES

### Example 1

In following example, the growth to harvest of seven diverse fragrant and six diverse non-fragrant rice varieties under three levels of salt treatment has been observed in an attempt to determine whether the loss of BADH2 function in fragrant rice varieties affects tolerance to salt stress. The effect of salt stress on BADH1 and BADH2 in mature seed from non-fragrant rice has also been studied to help determine whether these genes may play a stress response role in rice seed.

### Experimental Procedure

### Plant Materials

Seed was obtained from the Australian Plant DNA Bank Ltd (Lismore, NSW 2480, Australia). The seven fragrant varieties chosen for this study were: Basmati 370 (AC01-1001016, Dellmont (Lemont/Della) (AC04-1003719), Dragon's Eyeball 100 (AC04-1003713), Dumsorhk (AC04-1003784), Goolarah (Della/Kulu) (AC04-1003776). The six non-fragrant varieties were: Amaroo (Calrose/M7 (AC04-1003714), Calrose (Caloro/Calady) (AC04-1003785), Doongara (Calrose/Bluebelle/Jojutla) (AC01-1001029), Jarrah (M7/Somewake) (AC04-3798), Millin (M7/Somewake) (AC04-1003779) and Teqing (AC04-1003747).

### Plant Growth

Three trays, tray 1 labelled 0 mM, tray 2 labelled 17 mM and tray 3 labelled 22 mM, were filled with 22 500 ml pots. The pots contained equal parts peatmoss, Perlite (size P500) and Vermiculite (size 2) with the addition of Osmocote exact (macro nutrients plus some trace elements) 5 g/L, Micromax (trace element mix) 1 g/L and Dolomite (calcium, magnesium, pH buffer) 1 g/L. For each rice variety, five seeds were evenly spaced within a single filled post in each tray. Seven fragrant and six non-fragrant rice varieties were arranged randomly in pots within each of three trays. Trays were filled with 10 L water containing 30 mls Maxicrop liquid fertilizer (Multicrop Pty. Ltd., Bayswater, Victoria, Australia). Growth of seedlings in a glasshouse under controlled temperature conditions (25° C.-35° C.) was monitored for 14 days, at which point all but the two best-established seedlings in each pot were removed. Growth of all remaining plants under identical conditions was allowed to continue for 9 weeks. Salt treatment commenced after 9 weeks at which point the plants were healthy and well established. Plants in tray 1 continued to be grown with 10 L water containing 30 mls liquid fertilizer. Plants in tray 2 were grown using the original solution with the addition of NaCl stock solution such that the total concentration of additional NaCl was 17 mM. Plants in tray 3 were grown using the original solution with the addition of NaCl stock solution such that the total concentration of additional NaCl was 22 mM. The concentration of each of the salt solutions was monitored using an electrical conductivity meter. Upon anthesis all plants were tagged, at 40 days post-anthesis (dpa), plants (excluding roots) were removed and allowed to dry for two days. All seed was then collected and an initial wet weight measurement of plants (excluding roots) was taken. Remaining plant material was then dried completely and a dry weight measurement was taken. For each plant, seed number and mean seed mass was recorded.

Primer for qRTPCR

Oligonucleotide primers were designed using Primer Premier Version 5.0 (Premier Biosoft International, Palo Alto, Calif.). The sequences of the genes encoding BADH1 (accession AB001348), BADH2 (AB096083), actin (AB047313), glyceraldehyde-3-phosphate dehydrogenase (GAPDH) (NM_{—}001068440) and beta tubulin (NM_{—}184995) were obtained from the NCBI website (www.ncbi.nlm.nih.gov). All primers were designed to bind to exons adjacent to opposite ends of a single intron so that PCR products amplified from genomic and cDNA could be distinguished by size. All primers were designed to produce cDNA products of approximately 100 bp. BADH1 and BADH2 primers were designed in areas of low homology to avoid non-specific amplification. Primers were tested for specificity in genomic DNA and cDNA across a range of fragrant and non-fragrant rice varieties. From these results a single primer pair per gene was selected for use in qRTPCR (Table 1). qRTPCR was performed using genomic DNA to determine the relative efficiency of primer pairs. The efficiencies of the primer pairs targeting BADH1, BADH2, actin and GAPDH genes were determined to be almost identical. The primer pairs targeting actin, GAPDH and beta tubulin were then used in qRTPCR to assess consistency of the transcript levels of these genes across a range of fragrant and non-fragrant rice varieties. From these results actin transcript level as assessed by qRTPCR using a single, optimized primer pair (Table 1) was selected for normalization purposes.

### RNA Extraction and Analysis

Prior to all RNA extractions bench space and equipment was cleaned using RNaseZAP (SIGMA-ALDRICH Louis, Mo., USA). RNA was initially extracted from seed using TRIzol (Invitrogen, Carlsbad, Calif., USA) phenol/chloroform extraction followed by purification with a Qiagen RNeasy plant mini kit 20 (Qiagen GMbH, Germany). Approximately 100 mg seed consisting of two-three seeds from each of the two individuals for each variety at each salt treatment level was used per sample. Concentration of RNA in samples was determined using a Nanodrop ND-1000 (Nanodrop Technologies, Wilmington, Del., USA). RNA integrity was determined using an Agilent TNA 6000 Nano LabChip kit (Agilent Technologies, Santa Clara, Calif., USA) in conjunction with an Agilent 2100 Bioanalyzer (Agilent Technologies, Santa Clara, Calif., USA).

cDNA Preparation

Contaminating genomic DNA was digested by adding 1 μL RNase free recombinant DNase I (Roche Diagnostics GmBH, Mannheim, Germany) to 5 μg of extracted nucleic acid in 5 μL DNase I recombinant RNase buffer, 1 μL protector RNase inhibitor (Roche Diagnostics GmBH, Mannheim, Germany) and incubating at 35° C. for 20 mins. A 10 minute 70° C. incubation following the addition of 2 μL 0.2M EDTA (pH 8) stopped the reaction. RNA integrity was again tested post-DNase treatment using the Agilent 2100 Bioanalyzer. Samples possessing an RNA integrity number (RIN) (Agilent Technologies, Santa Clara, Calif., USA) greater than ‘5’ were considered acceptable for continued analysis. cDNA synthesis was performed using SuperScript III first strand synthesis SuperMix (Invitrogen, Carlsbad, Calif., USA) on 100 ng DNase digested RNA according to the manufacturers instructions. cDNA was then aliquoted and stored at −20° C.

qRTPCR and PCR

qRTPCR was performed with Platinum SYBR green qPCR supermix-UDG (Invitrogen, Carlsbad, Calif., USA). 5 μL Forward and reverse primers (1 μM each), 12 μL SYBR green qPCR SuperMix-UDG, 6 μL water and 2 μL cDNA were added to each reaction. A Corbett Rotor-Gene RG-3000 (Corbett Research, Australia) was used to cycle each reaction 35 times as follows: 15s denaturation at 94° C., 15s annealing at 57° C. and 30s polymerization at 72° C. All other PCR was performed using 0.2 μL Platinum Taq DNA Polymerase (Gibco BRL), 1 μL of genomic DNA or cDNA, 2.5 μL of 10× buffer (Gibco BRL), 1 μL of 50 mM MgCl₂ (Gibco BRL) 1 μL of dNTPs [5 mM], 2.5 μL forward and reverse primer mix (10 μM each), in a total volume of 25 μL. PCR was performed using a PerkinElmer, Gene Amp PCR system 9700. Cycling conditions were an initial denaturation of 94° C. for 2 min followed by 30 cycles of 5 s at 94° C., 5s at 55° C., 5 s at 72° C.; concluding with a final extension of 72° C. for 5 min. All PCR and qRTPCR products were analysed by electrophoresis in ethidium bromide stained (0.5 μg/ml) 1.0% agarose gels. DNA molecular weight marker XIII (Roche) was used to estimate fragment size.

### Results

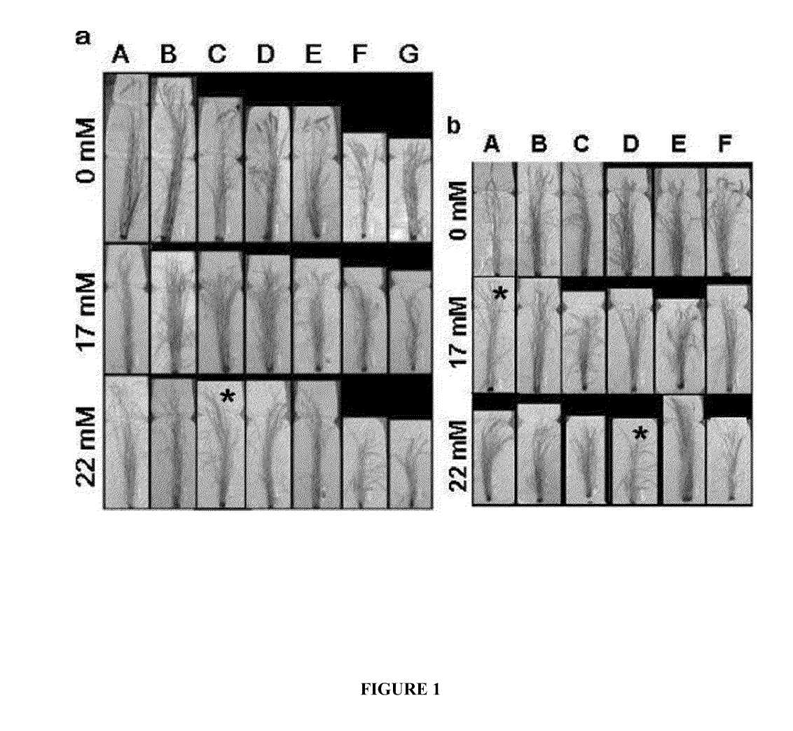

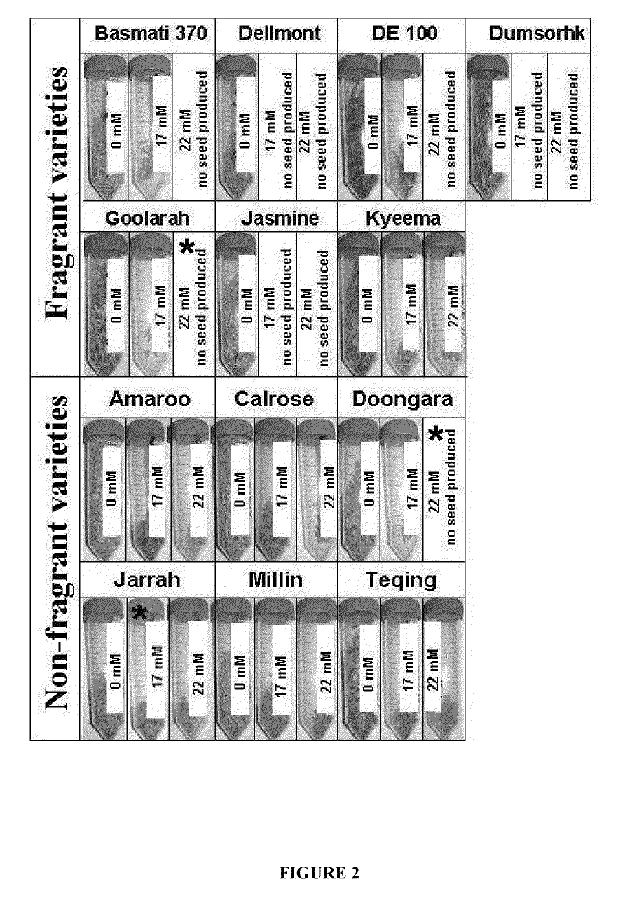

There was no significant difference between fragrant and non-fragrant varieties in wet mass or dry mass of the stem and leaf tissue at any of the salt concentrations (FIG. 1). Increasing salt concentration was associated with decreased wet and dry weight for fragrant and non-fragrant varieties (FIG. 2). Considerable variation between varieties was detected, however the average values for the fragrant and non-fragrant varieties were similar. One of the two individuals of the fragrant variety Goolarah and the non-fragrant variety Doongara planted for analysis under 22 mM salt treatment and the non-fragrant variety Jarrah planted for analysis under 17 mM salt treatment failed to thrive past the seedling stage so these data points were discounted. The single Goolarah and Jarrah plants that were grown conformed to the overall patterns in terms of seed production. Both Doongara individuals grown at 17 mM salt treatment showed very low seed production, the single plant grown at 22 mM failed to produce seed.

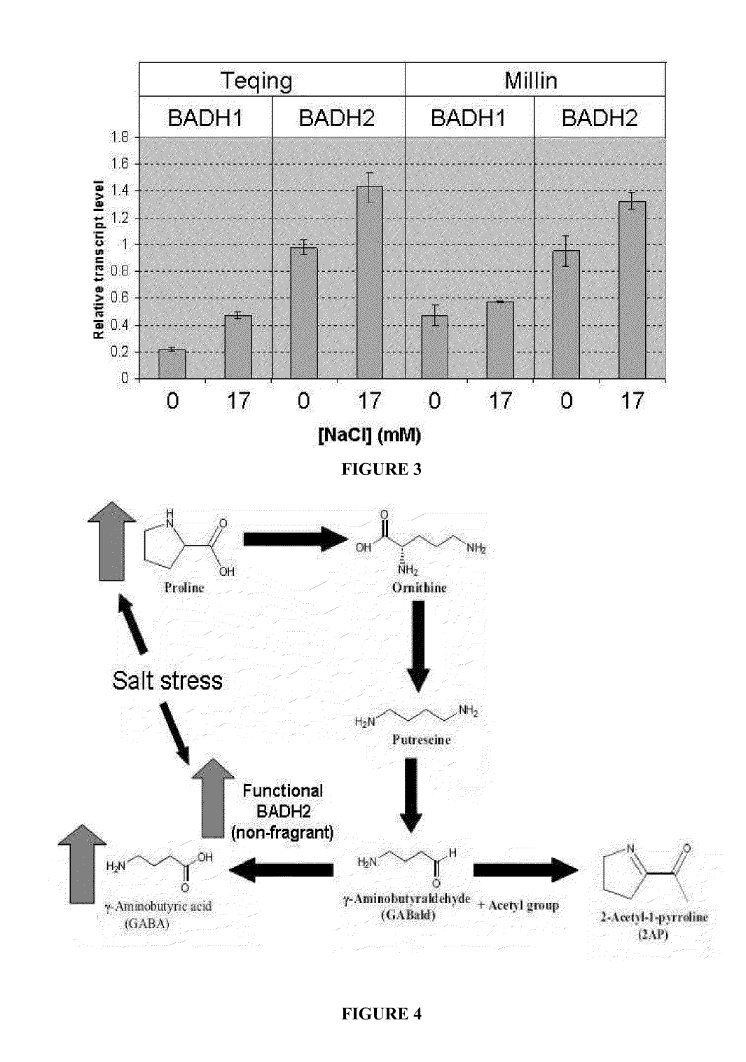

A strong relationship between fragrance phenotype and the ability of rice to produce mature seed under conditions of salt treatment were observed (Table 1, FIG. 3). For the plants grown with 0 mM additional salt, the fragrant varieties produced on average 31.5% less seed than the non-fragrant varieties (Table 1). For the plants grown at 17 mM additional salt, the fragrant varieties produced on average 92% less seed than the non-fragrant varieties (Table 1). At 17 mM additional salt, all non-fragrant varieties produced some seed, in contrast three of the seven fragrant varieties failed to produce any seed. For the plants grown at 22 mM additional salt, the fragrant varieties produced on average 97% less seed than the non-fragrant varieties (Table 1). At 22 mM additional salt, all but one non-fragrant variety (Doongara) produced some seed, in contrast all but one fragrant variety (Kyeema) failed to produce seed.

Mean transcript levels of both BADH1 and BADH2 from the non-fragrant rice varieties Millin and Teqing were significantly elevated in seed produced under growth conditions of 17 mM compared to that produced under control growth conditions (FIG. 3).

### Discussion

BADH genes have been shown to be involved in the abiotic stress response of many plant species, including cereal crops (Ishitani et al., 1997; Wood et al., 1996). In most plants BADH works in conjunction with CMO to produce glycine betaine, a compound with osmoprotectant properties. Despite not accumulating GB, rice possesses two BADH homologs and BADH1 has been shown to respond to salt treatment in leaf tissue in fragrant and non-fragrant rice varieties (Nakamura et al., 1997; Fitzgerald et al., Plant Sci., under review). Here we report that both BADH1 and BADH2 transcript levels are increased in mature seed from two non-fragrant rice varieties grown under conditions of salt treatment.

Fragrance in rice has been shown to result from the loss of function of a BADH2 encoding gene (Bradbury et al., 2005). BADH2 is known to be involved in the abiotic stress response in cereal species (Wood et al., 1996; Nakamura et al., 2001), however no loss of stress tolerance in fragrant rice varieties has previously been reported. The data presented here demonstrates that there is a strong link between the fragrance phenotype and the ability of rice to tolerate exposure to salt. It appears that loss of function of BADH2 which is responsible for rice fragrance also makes rice more susceptible to salt stress. The reduced vigour of fragrant rice varieties was not apparent during the vegetative phase of growth, but there was a very significant relationship between fragrance, salt exposure and seed production.

On the basis of previous gene expression studies (Nakamura et al., 1997; Fitzgerald et al., Plant Science, under review) and the growth and gene expression data presented here, it appears likely that both BADH1 and BADH2 are involved in the salt stress response in rice. BADH1 transcript levels increase in response to salt treatment in leaf and seed tissue from both fragrant and non-fragrant rice varieties, whilst BADH2 appears to respond in a tissue specific manner, with BADH2 transcript levels increasing in mature seed but not leaf tissue from two non-fragrant rice varieties (Millin and Teqing).

Glycine betaine accumulation has been the mechanism by which BADH and CMO have been thought to confer abiotic stress tolerance in those plants which accumulate this compound (Sakamoto and Murata, 2000). Since rice does not accumulate GB (Rathinasabapathi et al., 1993), BADH in rice must confer salt tolerance via a different mechanism. Bradbury et al. (Plant Mol. Biol., under review) have shown that the most preferable biochemical pathway for 2AP production is via gamma-aminobutyraldehyde (GABald). Bradbury proposed that BADH2 catalyses the formation of gamma-aminobutyric acid (GABA) from GABald. The ring form of GABald, delta-1-pyrroline, can combine with an acetyl group to form 2AP (Hoffman and Schieberle, 1998; Adams and De Kimpe, 2007) (FIG. 4). It is proposed that the absence of functional BADH2 leads to increased levels of delta-1-pyrroline and 2AP, and decreased levels of GABA. GABA is induced by abiotic stress in plants, recently Kim et al. (2007) have shown that GABA is strongly induced by salt stress in leaves from the non-fragrant rice variety Nipponbare. It appears plausible that the decrease in salt tolerance observed in fragrant rice varieties may be due to a decrease in the ability of fragrant rice varieties to accumulate GABA (FIG. 4). The loss of function of BADH2 might also lead to the accumulation of putrescine, a direct precursor to GABald and a substance known to be toxic to plants at elevated concentrations, which could also lead to decreased performance in fragrant rice. We have shown here that the loss of function of BADH2 appears to be responsible for a decreased ability of fragrant rice to produce mature seed when exposed to salt. It is known that fragrance is associated with significantly lower yields, but the reason for this has been unclear. Decreased tolerance to abiotic stress due to the lack of a functional BADH2 may, at least in part, explain this phenomenon. This observation further suggests that keeping abiotic stress to an absolute minimum, below the low level which is tolerated by non-fragrant varieties, may be a means of increasing yield in fragrant varieties.

Significant effort has been expended in attempt to develop fragrant rice varieties with increased yield under normal growth conditions. The data presented here suggests traditional fragrant rice varities are inherently less vigorous because the gene responsible for fragrance also leads to a loss of tolerance to salt and potentially other stresses. This has implications for attempts to produce higher yielding, more vigorous fragrant rice varieties using conventional breeding and may make transgenic approaches more appealing. For a transgenic approach to be successful, however, the biochemical pathway to 2AP production would have to remain undisturbed. The transformation of Basmati rice with a codA gene whose product catalyses the direct conversion of choline to glycine betaine provided increased salt tolerance in transgenic lines (Mohanty et al., 2002). Although the effect on 2AP concentration of the transformation with codA was not reported by Mohanty et al. (2002), codA would be unlikely to have significant affinity for GABald, so the pathway to the production of fragrance should not be affected by transgenic codA expression.

This discovery illustrates an excellent example of human selection for a trait which is deleterious in terms on plant vigour. In selecting for the non-functional BADH2 allele which imparts 2AP based rice fragrance, humans have propagated rice varieties which possess inferior tolerance to salt stress.

### SUMMARY

Salt stress results in the accumulation of proline in plants. A gene annotated as a betaine aldehyde dehydrogenase (BADH2) has been shown to contribute to proline metabolism by acting on 4-aminobutyraldehyde. In many plant species, BADHs play a role in abiotic stress tolerance, in this study mature seeds of non-fragrant rice plants subjected to salt treatment were found to have elevated levels of mRNA encoding BADH2 and BADH1 suggesting that both enzymes may be involved in salt tolerance. Fragrance in rice has been shown to result from deletions that cause the loss of function BADH2. To investigate whether the loss of function of this enzyme has an impact on the ability of rice to tolerate salt, the growth to harvest of seven diverse fragrant and six diverse non-fragrant rice varieties under salt treatment was studied. A strong effect of the fragrance phenotype on the ability of rice plants to produce mature seed was observed. When exposed to 17 mM NaCl from two months post-planting until harvest, the non-fragrant varieties produced on average a 10× greater number of seed per plant than the fragrant varieties. When exposed to 22 mM NaCl, the non-fragrant varieties produced 30× more seed per plant than the fragrant varieties. The apparent loss of stress tolerance in fragrant genotypes has major implications for breeding and production of the highly valued fragrant rices. This result may explain the relatively low yield of fragrant rice varieties and the restriction of much of the production to highly favourable environments. It suggests the possibility of improving yields of fragrant rice by breeding specifically for enhanced abiotic stress tolerance.

### Example 2

### Backcross Breeding and Salt Tolerance Screening for the Production of a High Yielding Fragrant Rice Variety by Backcrossing to a Salt Tolerant Non-Fragrant Parent

The rice cultivars Dellmont (salt-sensitive) and Pokkali (salt-tolerant) (Pattanagul and Thitisaksakul 2008) will be used as the parents for the purpose of generating a high yielding fragrant rice variety by backcrossing to a salt tolerant non-fragrant parent. Pokkali will be the female parent (pollen recipient) and Dellmont or the F₁, BC₁-BC₄ lines will be the male parent (pollen donor) for all crosses. At generations BC₁-BC₄ all plants carried forward in the breeding program will be genotyped using the assay of Bradbury et al (2005) to ensure they carriedfgr. In the first cross, ten Pokkali plants will be crossed with ten Dellmont plants. Mature F₁ seeds will be collected from the ten Pokkali parents, germinated and of these, ten F₁ plants will be crossed to ten cv Pokkali plants. Mature BC₁ seeds will be collected from the ten Pokkali parents, germinated and of these, ten BC₁ plants will be crossed to ten cv Pokkali plants. Mature BC₂ seeds will be collected from the ten Pokkali parents, germinated and of these, ten BC₂ plants will be crossed to ten cv Pokkali plants. Mature BC₃ seeds will be collected from the ten Pokkali parents, germinated and of these, ten BC₃ plants will be crossed to ten cv Pokkali plants. Mature BC₄ seeds will be collected from the ten Pokkali parents. These seeds will be germinated and carried through six generations of inbreeding to produce BC₄F₆ plants. At generation BC₄F₂, the plants will be genotyped using the assay of Bradbury et al (2005) and seed will be collected from those plants which are homozygous for fgr only.

One thousand single seeds will be collected from BC₄F₆ plants and these will be germinated and grown in the field for nine weeks under a standard rice plant management regime. At nine weeks post germination, the irrigation water will be drained from the field and flushed with water with a NaCl concentration of 22 mM. The water will be circulated through the field and the NaCl concentration monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

Seed from the 100 highest yielding BC₄F₇ lines will be germinated and grown in the field for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

Seed from the ten highest yielding BC₄F₈ lines will be germinated and grown in the field in a randomised block design for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

This procedure will be repeated over another two generations to ensure yield will be stably maintained. The three highest yielding lines re-entered the standard breeding program for further field testing. The line which attained the optimal balance between quality and yield will be preferably released as a cultivar.

### Example 3

### Backcross Breeding and Salt Tolerance Screening for the Production of a High Yielding Fragrant Rice Variety by Backcrossing to a Fragrant Parent

The rice cultivars Khao Dawk Mali 105 (salt-sensitive) and Pokkali (salt-tolerant) (Pattanagul and Thitisaksakul 2008) will be used as the parents for the purpose of generating a high yielding fragrant rice variety by backcrossing to a fragrant parent. Dellmont will be the female parent (pollen recipient) and Pokkali or the F₁, BC₁-BC₄ lines will be the male parent (pollen donor) for the crosses. At generations BC₁-BC₄ all plants carried forward in the breeding program will be genotyped using the assay of Bradbury et al (2005) to ensure they carried fgr. In the first cross, ten Pokkali plants will be crossed with ten Dellmont plants. Mature F₁ seeds will be collected from the ten Dellmont parents, germinated and of these, ten F₁ plants will be crossed to ten cv Dellmont plants. Mature BC₁ seeds will be collected from the ten Dellmont parents, germinated and of these, ten BC₁ plants will be crossed to ten cv Dellmont plants. Mature BC₂ seeds will be collected from the ten Dellmont parents, germinated and of these, ten BC₂ plants will be crossed to ten cv Dellmont plants. Mature BC₃ seeds will be collected from the ten Dellmont parents, germinated and of these, ten BC₃ plants will be crossed to ten cv Dellmont plants. Mature BC₄ seeds will be collected from the ten Dellmont parents. These seeds will be germinated and carried through six generations of inbreeding to produce BC₄F₆ plants. At generation BC₄F₂, the plants will be genotyped using the assay of Bradbury et al (2005) and seed will be collected from those plants which will be homozygous for fgr only.

One thousand single seeds will be collected from BC₄F₆ plants and these will be germinated and grown in the field for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

Seed from the 100 highest yielding BC₄F₇ lines will be germinated and grown in the field for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

Seed from the ten highest yielding BC₄F₈ lines will be germinated and grown in the field in a randomised block design for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

This procedure will be repeated for another two generations to ensure yield will be stably maintained. The three highest yielding lines re-entered the standard breeding program for further field testing. The line which attained the optimal balance between quality and yield will be preferably released as a cultivar.

### Example 4

### Pedigree Breeding and Salt Tolerance Screening for the Production of a High Yielding Fragrant Rice Variety

The rice cultivars Dellmont (salt-sensitive) and Pokkali (salt-tolerant) (Pattanagul and Thitisaksakul 2008) will be used as the parents for the purpose of generating a high yielding fragrant rice variety. Dellmont will be the female parent (pollen recipient) and Pokkali will be the male parent (pollen donor) for all crosses. Ten Pokkali plants will be crossed with ten Dellmont plants. Mature F₁ seeds will be collected from the ten Dellmont parents, germinated and carried through six generations of inbreeding to produce F₆ plants. At generation F₃, the plants will be genotyped using the assay of Bradbury et al (2005) and seed will be carried forward in the program from those plants which will be homozygous for fgr only.

One thousand single seeds will be collected from F₆ plants and these will be germinated and grown in the field for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

Seed from the 100 highest yielding F₇ lines will be germinated and grown in the field for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

Seed from the ten highest yielding F₈ lines will be germinated and grown in the field in a randomised block design for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

This procedure will be repeated over another two generations to ensure yield will be stably maintained. The three highest yielding lines re-entered the standard breeding program for further field testing. The line which attained the optimal balance between quality and yield will preferably be released as a cultivar.

### Example 5

### Molecular Breeding for the Production of a High Yielding Fragrant Rice Variety

The EMU promoter of the plasmid pEMUGN (Last et al., 1991) which consists of the EMU promoter and gus (uidA) gene and nos termination sequence, will be excised by restriction digestion. The linearised plasmid will be blunt ended and the maize ubiquitin gene (Ubi) promoter derived from pAHC18 (Bruce et al., 1989) inserted in its place.

The relevant protein coding sequences will be excised from any or all of the following plasmids, rice GS2 cDNA (Sakamoto et al, 1989), the full-length HVAZ cDNA (Hong et al, 1988), the PgNHX1 ORF (Accession No. DQ071264) (Verma et al, (2007) the full-length cDNA of SNAC1 (Hu et al, 2006) or the full-length TERF1 cDNA (Gao et al, 2008) and inserted 3′ to the UBI promoter and 5′ of gus of the modified pEMUGN plasmid to create the expression construct pSCUNatol1.

Transformation of the rice variety Basmati 370 with the expression construct pSCUNatol1 will be achieved following the protocol of Abedinia et al. (1997). Briefly, dehulled seeds (caryopsis) will be sterilized by rinsing in 70% ethanol followed by washing in 2% sodium hypochlorite and then sterile water. Callus induction medium (MSC), regeneration medium and medium for plantlet growth will be as described in Abedinia et al. (1997). Culture plates will be incubated in growth cabinet with 14 hour days at 27° C. and 10 hour nights of 22° C. A Dupont Biolistics PDS-1000/He (Bio-Rad) will be utilised to transform the rice callus. The gold particles will be coated with plasmid DNA according to Sanford et al. (1993) and bombardment conditions optimized using transient gus reporter gene assays (Jefferson, 1987). Plantlets of 10-15 cm will be transplanted and transferred to a glasshouse. Genomic DNA will be extracted using a Qiagen Dneasy® 96 Plant Kit (Qiagen GMbH, Germany). Transformed plants will be identified by PCR screening using primers for the gus gene as described in Abedinia et al. (1997) using the primers GUS-1 and GUS-2:

| | | |
|---|---|---|
| GUS-1 | 5′-GGTTGGGCAGGCCAGCGTATC-3′ | (SEQ ID NO: 1) |
| GUS-2 | 5′-CCAATGCCTAAAGAGAGGTTA-3′ | (SEQ ID NO: 2) |

Southern hybridization analysis will be used to determine the integration pattern of SSIIa. Genomic DNA will be digested with EcoRV and transferred onto Hybond N membrane (Amersham) according to the manufacturer's instructions. A ³²P-labeled PCR product generated by amplification of pSCUNatoll will be used as a probe for hybridization. Plants harbouring a single insertion of the target cDNA will be identified.

Seed from ten lines with single insertions of the target cDNA will be germinated and grown in the field in a randomised block design for nine weeks under a standard rice plant management regime. At nine weeks post germination, the irrigation water will be drained from the field and flushed with water with a NaCl concentration of 22 mM. The water will be circulated through the field and the NaCl concentration will be monitored using an electrical conductivity meter. At 40 days post-anthesis, the field will be drained and allowed to dry. The grain will be harvested and yield data consisting of yield and each of its components, panicles per plant, spikelets per panicle, grains per panicle and 1000-grain weight will be collected for each plant.

This procedure will be repeated for another two generations to ensure yield will be stably maintained. The three highest yielding lines re-entered the standard breeding program for further field testing. The line which attained the optimal balance between quality and yield will preferably be released as a cultivar.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein are incorporated herein by reference.

### REFERENCES

Adams, A., De Kimpe, N. (2007) Formation of pyrazines and 2-acetyl-1-pyrroline by Bacillus cereus. Food Chemistry 101, 1230-1238.
Bouché, N., Fromm, H. (2004) GABA in plants: just a metabolite? Trends in Plant Science 9, 110-115.
Bhatnagar-Mathur, P., Vadez, V., Sharma, K. K. (2008). Transgenic approaches for abiotic stress tolerance in plants: retrospect and prospects. Plant Cell Reports 27, 411-424.
Bradbury, L. M., Fitzgerald, T. L., Henry, R. J., Jin, Q., Waters, D. L. (2005) The gene for fragrance in rice. Plant Biotechnology Journal 3, 363-370.
Bradbury, L. M. E., Gillies, S. A., Brushett, D., Waters, D. L. E., Henry, R. J. (2008) Inactivation of an aminoaldehyde dehydrogenase is responsible for fragrance in rice. Plant Molecular Biology, in press.
Chen, S., Yang, Y., Shi, W., Ji, Q., He, F., Zhang, Z., Cheng, Z., Liu, X., Xu, M. (2008) Badh2, encoding betaine aldehyde dehydrogenase, inhibits the biosynthesis of 2-acetyl-1-pyrroline, a major component in rice fragrance. Plant Cell, in press.
Cordeiro, G. M., Christopher, M. J., Henry, R. J., Reinke, R. F. (2002) Identification of microsatellite markers for fragrance in rice by analysis of the rice genome sequence. Molecular Breeding 9, 245-250.
Eung-Jun, P., Zoran, J., Atsushi, S., Jeanine, D., Raweewan, Y., Norio, M., Tony, H. H. C. (2004) Genetic engineering of glycinebetaine synthesis in tomato protects seeds, plants, and flowers from chilling damage. Plant Journal 40, 474-487.
Fitzgerald, T. L., Waters, D. L. E., Henry, R. J. (2008) The effect of salt on betaine aldehyde dehydrogenase transcript levels and 2-acetyl-1-pyrroline concentration in fragrant and non-fragrant rice (Oryza sativa). Plant Science 174, 539-546
Garcia, A. B, Engler, J. D. A, Iyer, S., Gerats, T., Van Montagu, M., Caplan, A. B. (1997) Effects of osmoprotectants upon NaCl stress in rice. Plant Physiology 115, 159-169.
Gorham, J. Betaines in higher plants—biosynthesis and role in stress metabolism, in: Wallsgrove R M (Ed.), Amino Acids and their derivatives in higher plants, Cambridge: Cambridge University Press (1995), pp. 171-203.
Harinasut, P., Tsutsui, K., Takabe, T., Nomura, M., Takabe, T., Kishitani, S. (1996) Exogenous glycinebetaine accumulation and increased salt-tolerance in rice seedlings. Bioscience, biotechnology, and biochemistry 60, 366-368.
Hofmann, T., Schieberle, P. (1998) 2-oxopropanal, hydroxy-2-propanone, and 1-pyrroline: Important intermediates in the generation of the roast-smelling food flavour compounds 2-acetyl-1-pyrroline and 2-acetyltetrahydropyridine. Journal of Agriculture and Food Chemistry 46, 2270-2277.
Ishitani, M., Nakamura, T., Han, S. Y., Takabe, T. (1995) Expression of the betaine aldehyde dehydrogenase gene in barley in response to osmotic stress and abscisic acid. Plant Molecular Biology 27, 307-315.
Jin, Q., Waters, D., Cordeiro, G. M., Henry, R. J., Reinke, R. F. (2003) A single nucleotide polymorphism (SNP) marker linked to the fragrance gene in rice (Oryza sativa L.). Plant Science 165, 359-364.
Kim, D. W., Shibato, J., Agrawal, G. K., Fujihara, S., Iwahashi, H., Kim du, H., Shim Ie, S., Rakwal, R. (2007) Gene transcription in the leaves of rice undergoing salt-induced morphological changes (Oryza sativa L.). Molecules and Cells 24, 45-59.
Koyama, M. L., Levesley, A., Koebner, R. M. D, Flowers, T. J., Yeo, A. R. (2001) Quantitative trait loci for component physiological traits determining salt tolerance in rice. Plant Physiology 125, 406-422
Le Rudulier, D., Strom, A. R., Dandekar, A. M., Smith, L. T., Valentine, R. C. (1984) Molecular biology of osmoregulation. Science 224, 1064-1068.
Mohanty, A., Kathuria, H., Ferjani, A., Sakamoto, A., Mohanty, P., Murata, N., Tyagi, A. K. (2002) Transgenics of an elite indica rice variety Pusa Basmati 1 harbouring the codA gene are highly tolerant to salt stress. Theoretical and Applied Genetics 106, 51-57
Nakamura, T., Nomura, M., Mori, H., Jagendorf, A. T., Ueda, A., Takabe, T. (2001) An isozyme of betaine aldehyde dehydrogenase in barley. Plant and Cell. Physiology 42, 1088-1092.
Nakamura, T., Yokota, S., Muramoto, Y., Tsutsui, K., Oguri, Y., Fukui, K., Takabe, T. (1997) Expression of a betaine aldehyde dehydrogenase gene in rice, a glycinebetaine nonaccumulator, and possible localization of its protein in peroxisomes. The Plant Journal 11, 1115-1120.
Palanivelu, R., Brass, L., Edlund, A. F., Preuss, D. (2003) Pollen tube growth and guidance is regulated by POP2, an Arabidopsis gene that controls GABA levels. Cell 114, 47-59.
Rathinasabapathi, B., Gage, D. A., Mackill, D. J., Hanson, A. D. (1993) Cultivated and wild rices do not accumulate glycinebetaine due to deficiencies in two biosynthetic steps. Crop Science 33, 534-538.
Roychoudhury, A., Basu, S., Sarkar, S. N., Sengupta, D. N. (2008). Comparative physiological and molecular responses of a common aromatic indica rice cultivar to high salinity with non-aromatic indica rice cultivars. Plant Cell Reports 27, 1395-1410.
Sakamoto, A., Murata, N. (2000) Genetic engineering of glycinebetaine synthesis in plants: current status and implications for enhancement of stress tolerance. Journal of Experimental Botany 51, 81-88.
Shirasawa, K., Takabe, T., Takabe, T., Kishitani, S. (2006) Accumulation of glycinebetaine in rice plants that overexpress choline monooxygenase from spinach and evaluation of their tolerance to abiotic stress. Annals of Botany 98, 565-571.
Singh, R. K., Singh, U.S., Khush, G. S. (Eds.) Aromatic rices. Oxford & IBH Publishing Co. Pvt. Ltd., New Delhi (2000).
Ueda, A., Kathiresan, A., Inada, M., Narita, Y., Nakamura, T., Shi, W., Takabe, T., Bennett, J. (2004) Osmotic stress in barley regulates expression of a different set of genes than salt stress does. Journal of Experimental Botany 55, 2213-2218.
Widjaja, R., Craske, J. D., Wootton, M. (1996) Comparative studies on volatile components of non-fragrant and fragrant rices. Journal of the Science of Food and Agriculture 70, 151-161.
Wood, A. J., Saneoka, H., Rhodes, D., Joly, R. J., Goldsbrough, P. B. (1996) Betaine aldehyde dehydrogenase in sorghum. Plant Physiology 110, 1301-1308.
Yang, X., Liang, Z., Lu, C. (2005) Genetic engineering of the biosynthesis of glycinebetaine enhances photosynthesis against high temperature stress in transgenic tobacco plants. Plant Physiology 138, 2299-2309.
Bradbury, L. M. T., R. J. Henry, et al. (2005). “A perfect marker for fragrance genotyping in rice.” Molecular Breeding 16(4): 279-283.
Pattanagul, W. and M. Thitisaksakul (2008). Effect of salinity stress on growth and carbohydrate metabolism in three rice (Oryza sativa L.) cultivars differing in salinity tolerance. Indian Journal of Experimental Biology 46(10): 736-742.
Abedinia, M., Henry, R. J., Blakeney, A. B. and Lewin, L. (1997) An efficient transformation system for the Australian rice cultivar, Jarrah. Australian Journal of Plant Physiology. 24, 133-141
Sanford, J. C., Smith, F. D., and Russell, J. A. (1993) Optimizing the biolistic process fro different biological applications. Methods in Enzymology 217, 483-509
Jefferson, R. A. (1987). Assaying chimeric genes in plants: the GUS gene fusion system. Plant Molecular Biology Reporter. 5:387-405
Last, D. I., Brettell, R. I. S., Chamberlain, D. A., Chaudhury, A. M., Larkin, P. J., Marsh, E. L., Peacock, W. J. and Dennis, E. S. (1991) The use of the Emu promoter with antibiotic and herbicide resistance genes for the selection of transgenic wheat callus and rice plants. Theoretical and Applied Genetics 81:581-588
Bruce, W. B., Christensen, A. H., Klein, T., Fromm, M., and Quail, P. H. (1989) Photoregulation of a phytochrome gene promoter from oat transferred into rice by particle bombardment. Proceedings of the National Academy of the Sciences 86:9692-9696
Sakamoto, A., Ogawa, M., Masumura, T., Shibata, D., Takeba, G., Tanaka, K. and Fujii, S. 1989. Three cDNA sequences coding for glutamine synthetase polypeptides in Oryza sativa L. Plant Molecular Biology 13:611-614.
Hong B, Uknes S J, Ho T-HD (1988) Cloning and characterization of a cDNA encoding a mRNA rapidly induced by ABA in barley aleurone layers. Plant Molecular Biology 11:495-506
Verma D, Singla-Pareek S L, Rajagopal D, Reddy M K and Sopory S K (2007) Functional validation of a novel isoform of Na+/H+ antiporter from Pennisetum glaucum for enhancing salinity tolerance in rice. Journal of Biosciences. 32(3):621-628,
Hu H, Dai M, Yao J, Xiao B, Li X, Zhang O, and Xiong L (2006) Overexpressing a NAM, ATAF, and CUC(NAC) transcription factor enhances drought resistance and salt tolerance in rice. Proceedings of the National Academy of the Sciences 103 (35): 12987-12992
Gao S, Zhang H, Tian Y, Li F, Zhang Z, Lu X, Chen X, and Huang R (2008) Expression of TERF1 in rice regulates expression of stress-responsive genes and enhances tolerance to drought and high-salinity. Plant Cell Rep DOI 10.1007/s00299-008-0602-1

### TABLES

TABLE 1
Wet and dry mass of individual fragrant and non-fragrant rice varieties, and average values
for fragrants and non-fragrants, grown under three levels of salt treatment: 0 mM,
17 mM and 22 mM additional NaCl.

| | | plant | plant | | | | plant | plant | | | | plant | plant | |
|---|---|---|---|---|---|---|---|---|---|---|---|---|---|---|
| 0 mM | | 1 | 2 | mean | 17 mM | | 1 | 2 | mean | 22 mM | | 1 | 2 | mean |
| Goolarah | wet | 85 | 86 | 85.5 | Goolarah | wet | 95.1 | 92.4 | 93.75 | Goolarah | wet | 36.8 | * | 36.8 |
| | dry | 36.8 | 28.4 | 32.6 | | dry | 41.8 | 39.4 | 40.6 | | dry | 17.5 | * | 17.5 |
| Jasmine | wet | 60.5 | 50.2 | 55.35 | Jasmine | wet | 10.2 | 9 | 9.6 | Jasmine | wet | 7.6 | 4.5 | 6.05 |
| | dry | 10.9 | 10.4 | 10.65 | | dry | 4 | 3.6 | 3.8 | | dry | 6.4 | 2.4 | 3.9 |
| Dellmont | wet | 44.2 | 39.6 | 41.9 | Dellmont | wet | 33.2 | 38.5 | 35.85 | Dellmont | wet | 13.6 | 8.1 | 10.85 |
| | dry | 21.4 | 14.9 | 18.15 | | dry | 12.8 | 17.3 | 15.05 | | dry | 10.3 | 6.1 | 8.2 |
| Dumsorhk | wet | 65.6 | 78.4 | 72 | Dumsorhk | wet | 40.3 | 38.6 | 39.45 | Dumsorhk | wet | 47.6 | 43.4 | 45.5 |
| | dry | 23.5 | 30.2 | 26.85 | | dry | 16.4 | 14 | 15.2 | | dry | 24.1 | 21.3 | 22.7 |
| Kyeema | wet | 50.3 | 42.1 | 46.2 | Kyeema | wet | 42.1 | 46.3 | 44.2 | Kyeema | wet | 42 | 42.8 | 42.4 |
| | dry | 16.2 | 16.8 | 16 | | dry | 18.9 | 18.3 | 18.6 | | dry | 15.2 | 16.8 | 16 |
| Bas 370 | wet | 167.9 | 148.3 | 158.1 | Bas 370 | wet | 102.1 | 94.8 | 98.45 | Basmati 370 | wet | 101.3 | 95.8 | 98.55 |
| | dry | 72.5 | 67.3 | 69.9 | | dry | 40.8 | 38.9 | 39.85 | | dry | 47.7 | 47.5 | 47.6 |
| DE100 | wet | 79.8 | 81.9 | 80.85 | DE100 | wet | 94.8 | 53.4 | 74.1 | DE100 | wet | 30.3 | 18.1 | 24.2 |
| | dry | 31.6 | 31.9 | 31.75 | | dry | 32.1 | 20.1 | 26.1 | | dry | 16.3 | 11.1 | 13.7 |
| Calrose | wet | 120.2 | 101.9 | 111.05 | Calrose | wet | 132.4 | 112.6 | 122.5 | Calrose | wet | 130.4 | 110.3 | 120.35 |
| | dry | 55.9 | 42.9 | 49.4 | | dry | 49.7 | 46.8 | 47.75 | | dry | 46.7 | 46.7 | 46.2 |
| Amaroo | wet | 66.6 | 82.9 | 89.75 | Amaroo | wet | 75.3 | 67.5 | 71.4 | Amaroo | wet | 32.4 | 22.1 | 27.26 |
| | dry | 20.8 | 31.4 | 26.1 | | dry | 22.3 | 20.1 | 21.2 | | dry | 11.6 | 7.4 | 9.6 |
| Millin | wet | 61.4 | 44.3 | 52.85 | Millin | wet | 56.8 | 34.2 | 45.5 | Millin | wet | 32 | 29.6 | 30.8 |
| | dry | 8.2 | 24.3 | 16.25 | | dry | 19.8 | 12.4 | 16.1 | | dry | 15.3 | 14.2 | 14.75 |
| Jarrah | wet | 25.3 | 26.4 | 25.85 | Jarrah | wet | 24.6 | * | 24.6 | Jarrah | wet | 35.1 | 23.2 | 29.16 |
| | dry | 8.6 | 9.4 | 9 | | dry | 10.1 | * | 10.1 | | dry | 17.2 | 12.4 | 14.8 |
| Teqing | wet | 56.3 | 60.4 | 58.35 | Teqing | wet | 55.1 | 43.4 | 49.25 | Teqing | wet | 10 | 7.2 | 8.6 |
| | dry | 12.8 | 16.5 | 14.65 | | dry | 17.4 | 13 | 15.2 | | dry | 6 | 4.1 | 5.05 |
| Doongara | wet | 61.3 | 62.9 | 62.1 | Doongara | wet | 30.2 | 27.6 | 28.9 | Doongara | wet | 13.2 | * | 13.2 |

| | | | |
|---|---|---|---|
| | 0 mM | 17 mM | 22 mM |
| Fragrants wet | 77.1 | 56.6 | 37.8 |
| Fragrants dry | 29.4 | 22.7 | 18.5 |
| Non-fragrants wet | 63.3 | 57.0 | 38.2 |
| Non-fragrants dry | 23.3 | 20.3 | 16.2 |

TABLE 2
Mean seed number and seed weight from all fragrant and non-fragrant individuals
grown under three levels of salt treatment. Values for individual varieties and mean
values for each phenotype (fragrant/non-fragrant) displayed.

| | 0 mM additional salt | | 17 mM additional salt | | 22 mM additional salt | |
|---|---|---|---|---|---|---|
| | Mean Seed | Mean Weight | Mean Seed | Mean Weight | Mean Seed | Mean Weight |
| Variety | Number | (mg) | Number | (mg) | Number | (mg) |
| Fragrant varieties | | | | | | |
| Basmati 370 | 560 | 20 | 10 | 16 | 0 | N/A |
| Dellmont | 165 | 26 | 0 | N/A | 0 | N/A |
| Dragon's Eyeball 100 | 305 | 33 | 40 | 24 | 0 | N/A |
| Dumsorhk | 370 | 29 | 0 | N/A | 0 | N/A |
| Goolarah | 460 | 25 | 3 | 22 | 0* | N/A* |
| Jasmine | 520 | 25 | 0 | N/A | 0 | N/A |
| Kyeema | 390 | 28 | 25 | 18 | 8 | 18 |
| Fragrant mean | 435 | 27 | 13 | 20 | 1.5 | 18 |
| Non-fragrant varieties | | | | | | |
| Amaroo | 825 | 25 | 200 | 21 | 45 | 19 |
| Calrose | 730 | 24 | 205 | 23 | 10 | 21 |
| Doongara | 455 | 24 | 7 | 25 | 0* | N/A* |
| Jarrah | 290 | 28 | 95* | 26* | 28 | 24 |
| Millin | 425 | 28 | 220 | 22 | 35 | 15 |
| Teqing | 740 | 24 | 75 | 15 | 65 | 14 |
| Non-fragrant mean | 620 | 25.5 | 141 | 21 | 37 | 19 |

*Indicates that only a single individual grew past the seedling stage, these data points have been excluded in the calculation of the mean values.

1. A method of producing a fragrant plant with improved crop performance, said method including the step of propagating a fragrant plant with improved crop performance from a fragrant plant and a plant which has, or has been selected for, at least partial tolerance to one or more abiotic stress factors, to thereby produce a fragrant plant with improved crop performance.

2. The method of claim 1, wherein the fragrant plant does not have tolerance, or at least partial tolerance, to one or more abiotic stress factors.

3. The method of claim 2, wherein the one or more abiotic stress factors are selected from the group consisting of salt, drought, cold, freezing, high temperature, anoxia, high light intensity, nutrient imbalance, heavy metal tolerance and combinations thereof.

4. The method of claim 3, wherein the abiotic stress factor is salt.

5. The method of claim 1, wherein the plant or the fragrant plant is a monoctylednous plant or a dicotyledonous plant.

6. The method of claim 1, wherein the plant or the fragrant plant is selected from the group consisting of asparagus, bamboo, corn, barley, wheat, rice, sorghum, onion, pearl millet, rye, oats, rape, Zea mays, Zoysia tenuifolia, Musa acuminata, Pandan, tomato, beans, soybeans, peppers, lettuce, peas, alfalfa, cabbage, tobacco, broccoli, cauliflower, brussel sprouts, radish, carrot, beets, eggplant, spinach, cucumber, squash, sunflowers and combinations thereof.

7. The method of claim 6, wherein the plant or the fragrant plant is selected from the group consisting of wheat, rice, barley, oats, maize, Zea mays, sorghum, Zoysia tenuifolia and combinations thereof.

8. The method of claim 7, wherein the plant or the fragrant plant is rice.

9. The method of claim 8, wherein the rice variety is selected from the group consisting of jasmine, Basmati 370, Kyeema, Dellmont, Dragon's Eyeball 100, Dumsorhk, Khao Dawk Mali 105, Goolarah, Gobindobhog, Pokkali, KDML and combinations thereof.

10. A fragrant plant with improved crop performance produced according to a method of claim 1.

11. A method of producing a genetically-modified fragrant plant with improved crop performance, said method including the step of genetically modifying one or more fragrant plant cells or tissues to thereby produce a genetically-modified fragrant plant with improved crop performance, wherein said genetically-modified fragrant plant with improved crop performance retains at least a portion of a fragrance.

12. The method of claim 10, wherein the step of genetic modification is introduction of a heterologous nucleic acid into one or more fragrant plant cells or tissues.

13. The method of claim 11, wherein the step of genetically modifying one or more fragrant plant cells or tissues confers at least partial tolerance to one or more abiotic stress factors.

14. The method of claim 13, wherein the one or more abiotic stress factors are selected from the group consisting of salt, drought, cold, freezing, high temperature, anoxia, high light intensity, nutrient imbalance, heavy metal tolerance and combinations thereof.

15. The method of claim 14, where the abiotic stress factor is salt.

16. The method of claim 11, wherein the fragrant plant is selected from the group consisting of asparagus, bamboo, corn, barley, wheat, rice, sorghum, onion, pearl millet, rye, oats, rape, Zea mays, Zoysia tenuifolia, Musa acuminata, Pandan, tomato, beans, soybeans, peppers, lettuce, peas, alfalfa, cabbage, tobacco, broccoli, cauliflower, brussel sprouts, radish, carrot, beets, eggplant, spinach, cucumber, squash, sunflowers and combinations thereof.

17. The method of claim 16, wherein the fragrant plant is selected from the group consisting of wheat, rice, barley, oats, maize, Zea mays, sorghum, Zoysia tenuifolia and combinations thereof.

18. The method of claim 17, the fragrant plant is rice.

19. A genetically-modified fragrant plant with improved crop performance produced according to a method of claim 11.

20. A method of producing a fragrant plant with improved crop performance, said method including the steps of:
(i) introducing one or more mutations into genetic material of a fragrant plant; and
(ii) selecting a fragrant plant having one or more mutations which is at least partially tolerant to one or more abiotic stress factors, to thereby produce a fragrant plant with improved crop performance.

21. The method of claim 20, wherein the one or more abiotic stress factors are selected from the group consisting of salt, drought, cold, freezing, high temperature, anoxia, high light intensity, nutrient imbalance, heavy metal tolerance and combinations thereof.

22. The method of claim 21, wherein the abiotic stress factor is salt.

23. The method of claim 20, wherein the fragrant plant is selected from the group consisting of asparagus, bamboo, corn, barley, wheat, rice, sorghum, onion, pearl millet, rye, oats, rape, Zea mays, Zoysia tenuifolia, Musa acuminata, Pandan, tomato, beans, soybeans, peppers, lettuce, peas, alfalfa, cabbage, tobacco, broccoli, cauliflower, brussel sprouts, radish, carrot, beets, eggplant, spinach, cucumber, squash, sunflowers and combinations thereof.

24. The method of claim 23, wherein the fragrant plant is selected from the group consisting of wheat, rice, barley, oats, maize, Zea mays, sorghum, Zoysia tenuifolia and combinations thereof.

25. The method of claim 24, wherein the fragrant plant is rice.

26. A fragrant plant with improved crop performance produced according to a method of claim 20.

27. A genetically-modified fragrant plant with improved crop performance, wherein said genetically-modified fragrant plant is at least partially tolerant to one or more abiotic stress factors.

28. The genetically-modified fragrant plant of claim 27, wherein the one or more abiotic stress factors are selected from the group consisting of salt, drought, cold, freezing, high temperature, anoxia, high light intensity, nutrient imbalance, heavy metal tolerance and combinations thereof.

29. The genetically-modified fragrant plant of claim 28, wherein the abiotic stress factor is salt.

30. A method of producing a fragrant plant with improved crop performance, said method of including the step of propagating a fragrant plant with improved crop performance from at least two fragrant plants with improved crop performance produced according to a method of claim 1.

31. A fragrant plant with improved crop performance produced according to a method of claim 31.

32. A method of producing a fragrant plant with improved crop performance, said method of including the step of propagating a fragrant plant with improved crop performance from at least two fragrant plants with improved crop performance produced according to the method of claim 11.

33. A fragrant plant with improved crop performance produced according to a method of claim 32.

34. A method of producing a fragrant plant with improved crop performance, said method of including the step of propagating a fragrant plant with improved crop performance from at least two fragrant plants with improved crop performance produced according to the method of claim 20.

35. A fragrant plant with improved crop performance produced according to a method of claim 34.
